(19) United States
(12) Patent Application Publication
(10) Pub. No.: US 2023/0227496 A1
(43) Pub. Date: Jul. 20, 2023

(54) FLOW-THROUGH PROCESSES AND DEVICES FOR PURIFYING A TARGET MOLECULE

(71) Applicant: 3M INNOVATIVE PROPERTIES COMPANY, St. Paul, MN (US)

(72) Inventors: Andrew W. VAIL, Bayport, MN (US); Semra COLAK ATAN, St. Louis Park, MN (US); Jerald K. RASMUSSEN, Woodville, WI (US); Alexei M. VOLOSHIN, Woodbury, MN (US); Jonathan F. HESTER, Hudson, WI (US)

(21) Appl. No.: 17/995,999

(22) PCT Filed: Apr. 1, 2021
(86) PCT No.: PCT/IB2021/052739

§ 371 Date: Oct. 12, 2022

Related U.S. Application Data
(60) Provisional application No. 63/009,080, filed on Apr. 13, 2020.

(51) Int. Cl. C07K 1/18; C07K 1/36; C08G 18/66

(52) U.S. Cl. CPC C07K 1/18; C07K 1/36; C08G 18/6688

(57) ABSTRACT

Flow-through processes for purifying a target molecule (e.g., antibodies, enzymes, and hormones, particularly a monoclonal antibody) from a biological solution (e.g., a neutralized viral inactivation pool) in a sample that includes the target molecule, and devices for carrying out such processes.

### BACKGROUND

One of the most challenging areas of biopharmaceutical drug production is the filtration/purification of monoclonal antibodies, which are important for therapeutic uses for a variety of diseases including rheumatoid arthritis, Crohn's disease, hypercholesterolemia, and a variety of cancers. Traditional downstream processing of therapeutic antibodies consists of many steps. Purification schemes involve flow-through and bind-and-elute chromatography. Many of these processes require buffer exchanges, which enhance the performance to reduce impurities such as DNA, HCP, and target protein aggregates (HMW), while trying to mitigate product loss. For example, ion exchange chromatography processes typically are run in bind-and-elute modes and often require buffer exchanges, such as pH and salt adjustments. Condensing these chromatography steps into a multifunction ion exchange or hybrid device by reducing or eliminating the buffer exchange steps would reduce processing time and materials during protein purification. The biopharmaceutical industry has an increased interest in identifying continuous processing schemes, particularly flow-through processes, to help reduce processing times and steps.

### SUMMARY OF THE DISCLOSURE

The present disclosure provides flow-through processes for purifying a target molecule from a biological solution in a sample that includes the target molecule, and devices for carrying out such processes.

In one embodiment, the flow-through process for purifying a target molecule from a biological solution in a sample includes: optionally, contacting the sample with an anion exchange adsorptive depth filter (i.e., filter element); optionally, conducting a buffer exchange with the sample before and/or after contacting the sample with an anion exchange adsorptive depth filter; contacting the sample with a salt-tolerant anion exchange nonfibrous porous filter element (e.g., membrane); and immediately thereafter, contacting the sample with a cation exchange nonfibrous porous filter element (e.g., membrane); wherein the flow-through process includes one or two buffer exchanges, with no buffer exchange between the sample contacting the salt-tolerant anion exchange nonfibrous porous filter element and the cation exchange nonfibrous porous filter element.

In another embodiment, the flow-through process for purifying a target molecule from a biological solution in a sample includes: contacting the sample with an anion exchange adsorptive depth filter; immediately thereafter, contacting the sample with a salt-tolerant anion exchange nonfibrous porous filter element; and immediately thereafter, contacting the sample with a cation exchange nonfibrous porous filter element; wherein the flow-through process includes no buffer exchanges.

In yet another embodiment, the flow-through process for purifying a target molecule from a biological solution in a sample includes: contacting the sample with an anion exchange adsorptive depth filter; conducting a buffer exchange with the sample after contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In still another embodiment, the flow-through process for purifying a target molecule from a biological solution in a sample includes: conducting a buffer exchange with the sample; contacting the sample with a salt-tolerant anion exchange nonfibrous porous filter element; and immediately thereafter, contacting the sample with a cation exchange nonfibrous porous filter element.

In a further embodiment, the present disclosure provides a filter cartridge that includes a salt-tolerant anion exchange nonfibrous porous filter element as described herein and a cation exchange nonfibrous porous filter element as described herein, preferably positioned downstream from the salt-tolerant filter element.

As used herein, “alkyl” refers to a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 12 carbon atoms, 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof Unless otherwise indicated, the alkylene group typically has 1 to 30 carbon atoms. In some embodiments, the alkylene group has 1 to 20 carbon atoms, 1 to 12 carbon atoms, 1 to 10 carbon atoms, 1 to 6 carbon atoms, or 1 to 4 carbon atoms. In some embodiments, the alkylene is a linear saturated divalent hydrocarbon having from 1 to 12 carbon atoms, and in some embodiments, the alkylene is a branched saturated divalent hydrocarbon having from 3 to 12 carbon atoms, e.g., methylene, ethylene, propylene, 2-methylpropylene, pentylene, hexylene, 1,4-cyclohexylene, 1,4-cyclohexyldimethylene,and the like.

The term “aryl” refers to a monovalent group that is aromatic and, optionally, carbocyclic. The aryl has at least one aromatic ring. Any additional rings can be unsaturated, partially saturated, saturated, or aromatic. Optionally, the aromatic ring can have one or more additional carbocyclic rings that are fused to the aromatic ring. Unless otherwise indicated, the aryl groups typically contain from 6 to 30 carbon atoms. In some embodiments, the aryl groups contain 6 to 20, 6 to 18, 6 to 16, 6 to 12, or 6 to 10 carbon atoms. Examples of an aryl group include phenyl, tolyl, benzyl, phenethyl, naphthyl, 2-naphthylethyl, biphenyl, phenanthryl, and anthracyl.

“Hydrocarbyl” is inclusive of aryl and alkyl. “Hydrocarbylene” is inclusive of arylene and alkylene.

“(Hetero)hydrocarbyl” is inclusive of hydrocarbyl (alkyl and aryl) groups, and heterohydrocarbyl (heteroalkyl and heteroaryl) groups. Heterohydrocarbyl groups include one or more catenary (in-chain) heteroatoms, or one or more substituents comprising heteroatoms, such as oxygen, sulfur, or nitrogen atoms. Heterohydrocarbyl groups may optionally contain one or more catenary (in-chain) functional groups including ester, amide, urea, urethane, and carbonate functional groups. Unless otherwise indicated, the non-polymeric (hetero)hydrocarbyl groups typically contain from 1 to 60 carbon atoms, 1 to 40 carbon atoms, 1 to 20 carbon atoms, 1 to 10 carbon atoms, or 1 to 6 carbon atoms. Some examples of such heterohydrocarbyls as used herein include, but are not limited to, methoxyethyl, ethoxypropyl, propoxyethyl, 4-diphenylaminobutyl, 2-(2′-phenoxyethoxyl)ethyl, 3,6-dioxaheptyl, 3,6-dioxahexyl-6-phenyl, 2-imidazolyl, 3-furyl, and 3-indolmethyl.

“(Hetero)hydrocarbylene” is inclusive of hydrocarbylene (alkylene and arylene) groups, and heterohydrocarbylene (heteroalkylene and heteroarylene) groups. Heterohydrocarbylene groups include one or more catenary (in-chain) heteroatoms, or one or more substituents including heteroatoms, such as oxygen, sulfur, or nitrogen atoms. Heterohydrocarbylene groups may optionally contain one or more catenary (in-chain) functional groups including ester, amide, urea, urethane, and carbonate functional groups. Unless otherwise indicated, the non-polymeric (hetero)hydrocarbylene groups typically contain from 1 to 60 carbon atoms, 1 to 40 carbon atoms, 1 to 20 carbon atoms, 1 to 10 carbon atoms, or 1 to 6 carbon atoms. Some examples of such heterohydrocarbylenes as used herein include, but are not limited to, oxydiethylene, 3-thiabutylene, 3-diphenylaminobutylene, 2-(2′-phenoxyethyl)ethylene, 3,6-dioxaoctylene, 3,6-dioxahexyl-6-phenylene, 2,5-furylene, 2,6-pyridylene (also known as 2,6-pyridinediyl), and 2,5-thiophenedimethylene.

The term “ethylenically unsaturated group” refers to those groups having carbon-carbon double (or triple) bonds that may be free-radically polymerized, and includes (meth)acrylamides, (meth)acrylates, vinyl and vinyloxy groups, allyl and allyloxy groups, and acetylenic groups.

The terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

Herein, the term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more preferred claims does not imply that other claims are not useful and is not intended to exclude other claims from the scope of the disclosure.

In this application, terms such as “a,” “an,” and “the” are not intended to refer to only a singular entity, but to include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the term “room temperature” refers to a temperature of 20° C. to 25° C. or 22° C. to 25° C.

The term “in the range” or “within a range” (and similar statements) includes the endpoints of the stated range.

Groupings of alternative elements or embodiments disclosed herein are not to be construed as limitations. Each group member may be referred to and claimed individually or in any combination with other members of the group or other elements found therein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

When a group is present more than once in a formula described herein, each group is “independently” selected, whether specifically stated or not. For example, when more than one Y group is present in a formula, each Y group is independently selected. Furthermore, subgroups contained within these groups are also independently selected. For example, when each Y group contains an R, then each R is also independently selected.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

The above summary of the present disclosure is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF THE FIGURES

FIG. 1(A-D) are flow diagrams representative of various flow-through processes for purifying a target molecule from a biological solution as described herein.

FIG. 2(A-C) are schematic figures of representative filter cartridge devices for carrying out various flow-through processes for purifying a target molecule from a biological solution as described herein.

### DETAILED DESCRIPTION OF ILLUSTRATIVE EMBODIMENTS

The present disclosure provides flow-through processes for purifying a target molecule from a biological solution in a sample that includes the target molecule, and devices for carrying out such processes.

In certain embodiments, the target molecules include therapeutic agents. In certain embodiments, the target molecules include viral vectors, proteins, such as antibodies and enzymes, hormones. In certain embodiments, the target molecule includes a monoclonal antibody.

In certain embodiments the biological solution includes a neutralized viral inactivation pool. Viral inactivation pool is a biological solution consisting of Protein A chromatography eluate, or combination of several Protein A eluates, that are then subjected to the viral inactivation process, which involves titration with acid followed by a hold time at the appropriate hold pH, typically pH≤3.5.

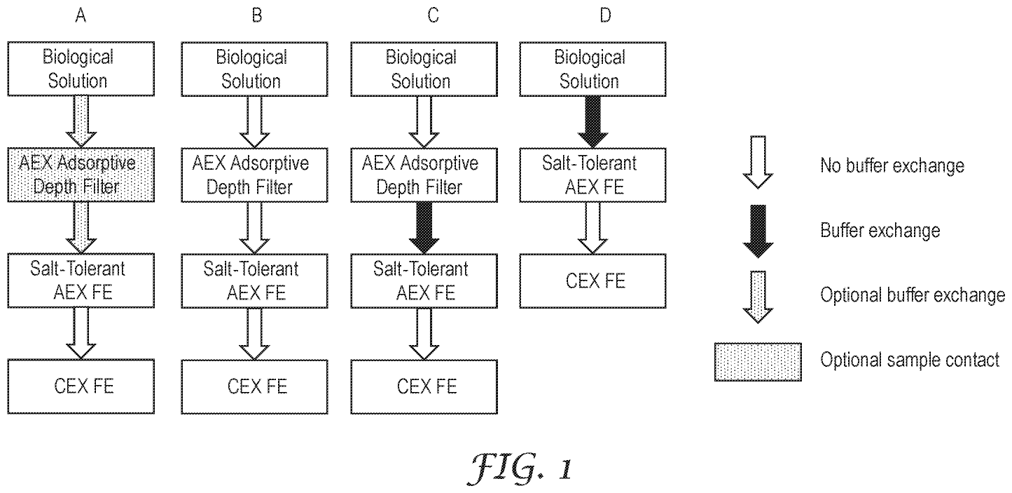

In one embodiment, the present disclosure provides a flow-through process for purifying a target molecule from a biological solution in a sample. As shown in FIG. 1A, the process includes: optionally, contacting the sample (of a biological solution) with an anion exchange adsorptive depth filter (AEX Adsorptive Depth Filter); optionally, conducting a buffer exchange with the sample before and/or after contacting the sample with an anion exchange adsorptive depth filter; contacting the sample with a salt-tolerant anion exchange nonfibrous porous filter element (Salt-Tolerant AEX FE); and immediately thereafter, contacting the sample with a cation exchange nonfibrous porous filter element (CEX FE) (e.g., membrane); wherein the flow-through process includes one or two buffer exchanges, with no buffer exchange between the sample contacting the salt-tolerant anion exchange nonfibrous porous filter element (e.g., membrane) and the cation exchange nonfibrous porous filter element.

Nonfibrous porous filter elements (e.g., membranes) and fibrous media formats allow high flow rates and good adsorption capacities but vary in resolving capabilities. For example, the smaller pore sizes of the nonfibrous porous filter elements and the better control of the pore size distribution provides better control in separation of impurities, particularly biological molecules. Also, nonfibrous porous filter elements can provide higher ion exchange capacities due to higher surface areas. Thus, nonfibrous porous filter elements may offer advantages over fibrous media at certain points in a flow-through purification process.

In the context of the present disclosure, “immediately thereafter” means that there is no other separation steps/media used between the recited two media.

In the context of the present disclosure, “flow-through” means that the target molecules pass through all filters.

The use of buffer exchanges advantageously optimizes interactions between impurities and membranes, and provides higher target yield from compositions with low impurity levels; however, reducing the number of buffer exchanges can reduce processing timing and costs.

In this context, “exchange” does not require changing the buffer. Although a buffer exchange may include a complete change in buffers (i.e., change of one buffer for another buffer) by known methods (e.g., Tangential Flow Filtration or Crossflow Filtration), an “exchange” can also include modifying a buffer by, e.g., changing the pH, changing the conductivity, and/or diluting the sample of interest. It may also be referred to as a buffer change or a buffer adjustment.

A “buffer” is a buffered solution that resists changes in pH by the action of an organic acid-base conjugate components.

In one embodiment, an organic acid (in a buffer) includes, but is not limited to, formic acid, acetic acid, lactic acid, citric acid, malic acid, maleic acid, glycine, phosphoric acid, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid).

In one embodiment, an organic base (in a buffer) includes, but is not limited to, the group consisting of tris base, arginine, Bis-Tris, Bis-Tris-Propane, Bicine (N,N-bis(2-hydroxyethyl)glycine), HEPES (4-2-hydroxyethyl-1-piperazineethane sulfonic acid), TAPS (3-{[tris(hydroxymethyl)methyl]amino}propanesulfonic acid), and Tricine (N-tris(hydroxymethyl)methylglycine).

In one embodiment, a conjugate base of an organic acid is the sodium, potassium, or ammonium salt of a conjugate base of an organic acid. In one embodiment, the organic acid is acetic acid and the conjugate base of acetic acid is the sodium salt.

Typically, buffers include equilibrium buffers, loading buffers, elution buffers, and the like. An “equilibration buffer” herein is that used to prepare a solid phase for chromatography. A “loading buffer” is that which is used to load a mixture of a protein and contaminant(s) onto a chromatography matrix. The equilibration and loading buffers can be the same. An “elution buffer” is used to elute proteins from the chromatography matrix.

In certain embodiments, the flow-through process includes: contacting the sample (of a biological solution) with an anion exchange adsorptive depth filter; optionally, conducting a buffer exchange with the sample before and/or after contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In certain embodiments, the flow-through process includes: contacting the sample (of a biological solution) with an anion exchange adsorptive depth filter; conducting a buffer exchange with the sample before and/or after contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In certain embodiments, the flow-through process includes: contacting the sample (of a biological solution) with an anion exchange adsorptive depth filter; conducting a buffer exchange with the sample after contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In certain embodiments, the flow-through process includes: contacting the sample with an anion exchange adsorptive depth filter; conducting a buffer exchange with the sample before contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In certain embodiments, the flow-through process includes: contacting the sample with an anion exchange adsorptive depth filter; conducting a buffer exchange with the sample before and after contacting the sample with an anion exchange adsorptive depth filter; contacting the sample with a salt-tolerant anion exchange nonfibrous porous filter element; and immediately thereafter, contacting the sample with a cation exchange nonfibrous porous filter element.

In certain embodiments, the flow-through process includes: conducting a buffer exchange with the sample after contacting the sample with a cation exchange nonfibrous porous filter element.

In certain embodiments, as shown in FIG. 1B, the flow-through process includes: contacting the sample (of a biological solution) with an anion exchange adsorptive depth filter (AEX Adsorptive Depth Filter); immediately thereafter, contacting the sample with a salt-tolerant anion exchange nonfibrous porous filter element (Salt-Tolerant AEX FE); and immediately thereafter, contacting the sample with a cation exchange nonfibrous porous filter element (CEX FE); wherein the flow-through process includes no buffer exchanges.

In certain embodiments, as shown in FIG. 1C, the flow-through process includes: contacting the sample (of a biological solution) with an anion exchange adsorptive depth filter (AEX Adsorptive Depth Filter); conducting a buffer exchange with the sample after contacting the sample with an anion exchange adsorptive depth filter; contacting the sample with a salt-tolerant anion exchange nonfibrous porous filter element (Salt-Tolerant AEX FE); and immediately thereafter, contacting the sample with a cation exchange nonfibrous porous filter element (CEX FE).

In certain embodiments, as shown in FIG. 1D, the flow-through process includes: conducting a buffer exchange with the sample (of a biological solution); contacting the sample with a salt-tolerant anion exchange nonfibrous porous filter element (Salt-Tolerant AEX FE); and immediately thereafter, contacting the sample with a cation exchange nonfibrous porous filter element (CEX FE).

### Optional Anion Exchange Adsorptive Depth Filter Elements

Depth filters (i.e., filter elements) are porous materials that allow particles (i.e., particulate) that can be in, for example, a viral inactivation pool, to penetrate and subsequently become trapped therein. That is, a depth filter captures contaminants within a sample between the upstream surface and a downstream surface of the filter substrate.

Exemplary depth filters include a porous base substrate and a grafted copolymer that includes interpolymerized cationic nitrogen-containing ligand monomers (i.e., ligands) as disclosed in U.S. Pat. No. 9,821,276 (Berrigan et al.), or grafted ligand-functional polymers as disclosed in U.S. Pat. No. 8,846,203 (Bothof et al.).

Porous Base Substrate. The porous substrate (i.e., base substrate) of the depth filter can be porous membranes, porous nonwoven webs, or porous fibrous substrate.

In certain embodiments, it is a porous nonwoven web. As used herein, the terms “nonwoven web” or “nonwoven substrate” are used interchangeably and refer to a fabric that has a structure of individual fibers or filaments which are randomly and/or unidirectionally interlaid in a mat-like fashion.

A fibrous nonwoven web can be made by carded, air laid, spunlaced, spunbonding or melt-blowing techniques or combinations thereof. Spunbonded fibers are typically small diameter fibers that are formed by extruding molten thermoplastic polymers as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g. air) stream which attenuates the filaments of molten thermoplastic material to reduce their diameter. Thereafter, the meltblown fibers are carried by the high velocity gas stream and are deposited on a collecting surface to from a web of randomly disbursed meltblown fibers. Any of the nonwoven webs may be made from a single type of fiber or two or more fibers that differ in the type of thermoplastic polymer and/or thickness.

Suitable nonwoven substrates for the depth filter may be spunlaid, hydroentangled, or meltblown. In certain embodiments, they may have a tensile strength of at least 4.0 newtons prior to grafting, a surface area of 15 to 50 m² per square meter of nonwoven substrate, a mean pore size of 1-40 microns according to ASTM F 316-03, and a solidity of less than 20%.

The porous base substrate may be formed from any suitable thermoplastic polymeric material. Suitable polymeric materials include, but are not limited to, polyolefins, poly(isoprenes), poly(butadienes), fluorinated polymers, chlorinated polymers, polyamides, polyimides, polyethers, poly(ether sulfones), poly(sulfones), poly(vinyl acetates), copolymers of vinyl acetate, such as poly(ethylene)-co-poly(vinyl alcohol), poly(phosphazenes), poly(vinyl esters), poly(vinyl ethers), poly(vinyl alcohols), and poly(carbonates).

Suitable polyolefins include, but are not limited to, poly(ethylene), poly(propylene), poly(l-butene), copolymers of ethylene and propylene, alpha olefin copolymers (such as copolymers of ethylene or propylene with 1-butene, 1-hexene, 1-octene, and 1-decene), poly(ethylene-co-1-butene) and poly(ethylene-co-1-butene-co-1-hexene).

Suitable fluorinated polymers include, but are not limited to, poly(vinyl fluoride), poly(vinylidene fluoride), copolymers of vinylidene fluoride (such as poly(vinylidene fluoride-co-hexafluoropropylene), and copolymers of chlorotrifluoroethylene (such as poly(ethylene-co-chlorotrifluoroethylene).

Suitable polyamides include, but are not limited to, poly(iminoadipoyliminohexamethylene), poly(iminoadipoyliminodecamethylene), and polycaprolactam. Suitable polyimides include, but are not limited to, poly(pyromellitimide).

Suitable poly(ether sulfones) include, but are not limited to, poly(diphenylether sulfone) and poly(diphenylsulfone-co-diphenylene oxide sulfone).

Suitable copolymers of vinyl acetate include, but are not limited to, poly(ethylene-co-vinyl acetate) and such copolymers in which at least some of the acetate groups have been hydrolyzed to afford various poly(vinyl alcohols).

In some embodiments, the porous base substrate is formed from a propylene homo- or copolymers, most preferably propylene homopolymers.

Immobilized Cationic Nitrogen-Containing Ligands. In certain embodiments, the porous substrate of the depth filter includes a grafted copolymer that includes interpolymerized cationic nitrogen-containing ligand monomers (i.e., ligands), as disclosed in U.S. Pat. No. 9,821,276 (Berrigan et al.). Alternatively stated, the porous substrate of the depth filter includes a copolymer grafted thereto (thereby forming a copolymer grafted article), wherein the grafted copolymer includes interpolymerized monomer units including cationic nitrogen-containing ligands.

In certain embodiments, the anion exchange ligands include cationic nitrogen-containing ligands. In certain embodiments, the cationic nitrogen-containing ligands include a primary amine, a secondary amine, a tertiary amine, or combinations thereof In certain embodiments, the cationic nitrogen-containing ligands include a quaternary ammonium-containing ligand, a guanidinyl-containing ligand, or a combination thereof.

In certain embodiments, the grafted copolymer includes interpolymerized monomer units including: a cationic nitrogen-containing ligand monomer selected from the group of quaternary ammonium-containing ligand monomers, guanidinyl-containing ligand monomers, and combinations thereof; an amide monomer; an oxy monomer selected from the group of epoxy functional monomer units, alkyl ether functional monomer units, and combinations thereof; and a poly(alkylene oxide) monomer.

In certain embodiments, the grafted copolymer includes interpolymerized monomer units including: 10 to 50 parts by weight of a cationic nitrogen-containing ligand monomer, wherein the cationic nitrogen-containing ligand monomer is selected from the group of quaternary ammonium-containing ligand monomers, guanidinyl-containing ligand monomers, and combinations thereof; 10 to 80 parts by weight of an amide monomer; 10 to 40 parts by weight of an oxy monomer selected from the group of epoxy functional monomer units, alkyl ether functional monomer units, and combinations thereof; and 0 to 30 parts by weight of a poly(alkylene oxide) monomer; wherein the sum of the monomers is 100 parts by weight.

In certain embodiments, the cationic nitrogen-containing ligand monomer is of the formula (I):

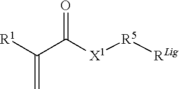

wherein:

R¹ is H or CH₃;

X¹ is —O— or —NR³—, where R³ is H or (hetero)hydrocarbyl;

R⁵ is a (hetero)hydrocarbylene; and

R^{Lig} is a quaternary ammonium ligand group or a guanidinyl-containing ligand group.

In certain embodiments, the cationic nitrogen-containing ligand monomer is a quaternary ammonium monomer (salt) of the formula (II):

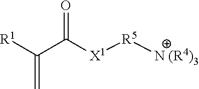

wherein:

R¹ is H or CH₃;

X¹ is —O— or —NR³—, where R³ is H or (hetero)hydrocarbyl;

R⁵ is a (hetero)hydrocarbylene; and

each R⁴ is independently alkyl or aryl.

The counterions of the quaternary ammonium salts include halides, sulfates, phosphates, nitrates, and the like. Exemplary quaternary ammonium salt monomers include (meth)acrylamidoalkyltrimethylammonium salts and (meth)acryloxyalkyltrimethylammonium salts, are described in U.S. Pat. No. 9,821,276 (Berrigan et al.).

In certain embodiments, the cationic nitrogen-containing ligand monomer is a guanidinyl-containing ligand monomer of the formula (III) or (IV):

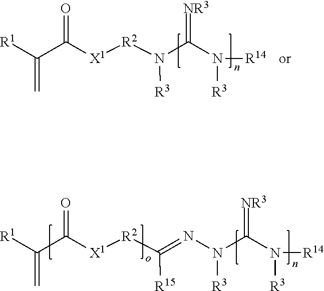

wherein:

R¹ is H or CH₃;

R² is a (hetero)hydrocarbylene (e.g., having 1 to 20 carbon atoms);

each R³ is independently H or (hetero)hydrocarbyl;

R¹⁴ is H, (hetero)hydrocarbyl, or —N(R³)₂, where each R³ is independently H or (hetero)hydrocarbyl;

R¹⁵ is H or hydrocarbyl (e.g., a C₁-C₄ alkyl group, or an aryl group);

X¹ is —O— or —NR³—, where R³ is H or (hetero)hydrocarbyl;

o is 0 or 1; and

n is 1 or 2.

Examples of cationic nitrogen-containing ligand monomers include agmatine-containing ligands (e.g., isocyanatoethylmethacrylate-agmatine adduct), guanidine-containing ligands, biguanide-containing ligands, and combinations thereof, prepared as described in U.S. Pat. No. 9,821,276 (Berrigan et al.).

In certain embodiments, the copolymer grafted article includes interpolymerized amide monomers ((meth)acrylamides and N-vinyl amides) of the formulas (V) or (VI):

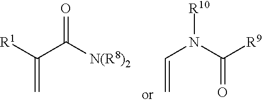

wherein:

R¹ is H or CH₃;

each R⁸ is independently hydrogen, alkyl, or aryl; and

R⁹ and R¹⁰ are alkyl groups, or may be taken together to form a 5 or 6-membered ring.

Examples of such monomers include N-vinyl caprolactam, N-vinyl acetamide, N-vinyl pyrrolidone, acrylamide, mono- or di-N-alkyl substituted acrylamide, and combinations thereof. In certain embodiments, the copolymer grafted article includes interpolymerized oxy monomers (epoxy-functional and monoether-functional (meth)acrylates and (meth)acrylamides) of the formula (VII):

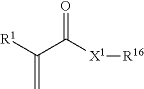

wherein:

R¹ is H or CH₃;

X¹ is —O— or —NR³—, where R³ is H or (hetero)hydrocarbyl; and

R¹⁶ is an epoxy-functional or ether-functional hydrocarbyl group.

In certain embodiments, the copolymer grafted article includes interpolymerized oxy monomers (epoxy monomers) of the formula (VIII):

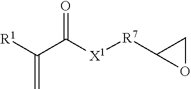

wherein:

R¹ is H or CH₃;

X¹ is —O— or —NR³—, where R³ is H or (hetero)hydrocarbyl; and

R⁷ is a (hetero)hydrocarbylene (e.g., a C₁-C₆ alkylene).

Examples of such monomers include glycidyl (meth)acrylate, thioglycidyl (meth)acrylate, 3-(2,3-epoxypropoxy)phenyl (meth)acrylate, 2-[4-(2,3-epoxypropoxyl)phenyl]-2-(4-(meth)acryloyloxy-phenyl)propane, 4-(2,3-epoxypropoxyl)cyclohexyl (meth)acrylate, 2,3-epoxycyclohexyl (meth)acrylate, and 3,4-epoxycyclohexyl (meth)acrylate, as well as others described in U.S. Pat. No. 9,821,276 (Berrigan et al.).

As described in U.S. Pat. No. 9,821,276 (Berrigan et al.), functionalized substrates may be prepared using the above-described monomers in a single reaction step or sequential reaction steps to provide a grafted polymer on the surface of a porous base substrate.

Grafted Ligand-Functional Polymers. In certain embodiments, the porous substrate of the depth filter includes a ligand-functional polymer grafted thereto, as disclosed in U.S. Pat. No. 8,846,203 (Bothof et al.).

In certain embodiments, the grafted ligand-functional polymer is of the formula (IX):

-(M^{PI})_{w}-(M^{b})ₓ-(M^{c})_{y}(M^{d})_{z}

wherein:

-(M^{PI})_{w} represent the residue of grafted photoinitiator monomers, where w is zero or at least one;

-(M^{b})ₓ represents polymerized ligand monomers, having “x” polymerized monomer units, where x is at least one;

-(M^{c})_{y} represents polymerized crosslinking monomers, having y polymerized monomer units, where y may be zero or at least one; and

-(M^{d})_{z} represents polymerized hydrophilic monomers, having z polymerized monomer units, where z may be zero or at least one.

In certain embodiments, the functionalized substrate has grafted groups attached to the surfaces of the base substrate that includes: a) optionally, at least one photoinitiator group (or the reaction product thereof); with b) one or more ligand monomers; c) optionally, one or more monomers having at least one acryloyl group and at least one additional free-radically polymerizable group; and d) optionally, one or more hydrophilic monomers.

The monomers that are grafted to the surface of the base substrates usually have both an acryloyl group for grafting by e-beam and at least one additional functional group thereon. Acryloyl groups, including acrylate and acrylamide groups, are preferred for direct grafting of the monomer to the substrate surface due to the greater reactivity of such acryloyl groups on exposure to ionizing radiation, such as e-beam irradiation. Not all such acryloyl groups may be “directly grafted,” i.e., forming a covalent bond with the substrate surface. Some may remain free and are subsequently “indirectly grafted” by incorporation into the polymer chain on exposure to UV radiation. Other ethylenically unsaturated groups, such as methacrylamides, methacrylates, vinyl and vinyloxy groups, allyl and allyloxy groups, and acetylenic groups are less reactive during e-beam grafting and are less likely to be directly grafted to the base substrate. Therefore, a portion of such non-acryloyl groups may be directly grafted, but largely remain unreacted, and are indirectly grafted to the substrate by incorporation into the polymer chain during UV initiated polymerization.

The photoinitiator “a)” monomers may be directly grafted onto surface of the base substrate, including the interstitial and outer surfaces of the porous base substrate to provide the grafted photoinitiator group via the acryloyl group.

The ligand “b)” monomers may have an acryloyl group for direct grafting or a non-acryloyl group, such as a methacrylate group, for subsequent incorporation (indirect grafting) into the polymer chain during UV initiated polymerization.

The acryloyl group of the “c)” monomers typically can directly graft (i.e. forming a covalent bond) to the surface of the base substrate when exposed to an ionizing radiation preferably e-beam or gamma radiation. In addition to an acryloyl group, the free-radically polymerizable groups of monomer “c)” are typically other ethylenically unsaturated groups such as methacrylamides, methacrylates, vinyl groups, and acetylenic groups having reduced reactivity during grafting, and are therefore free and unreacted for the subsequent UV initiated polymerization and crosslinking.

A fourth grafting hydrophilic monomer “d)” may also be grafted via an acryloyl group and may provide hydrophilic groups or ionic groups to the surfaces of the base substrate. In some embodiments, hydrophilic monomers having an ionic group may be directly or indirectly grafted to the substrate surface to provide secondary ionic interaction of the functionalized substrate.

The grafting photoinitiator monomers (M^{PI}) include an acryloyl group and a photoinitiator group, which may be a hydrogen-abstracting type or an α-cleavage-type photoinitiator group. Such grafting photoinitiator monomers (M^{PI}) are disclosed in U.S. Pat. No. 8,846,203 (Bothof et al.).

In certain embodiments, the ligand monomer (M^{b}) is of formula (X):

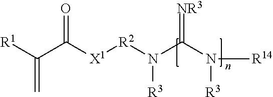

wherein:

R¹ is H or CH₃;

R² is a (hetero)hydrocarbylene;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl, and

n is 1 or 2.

In certain embodiments, the ligand monomer (M^{b}) is of the formula (XIV):

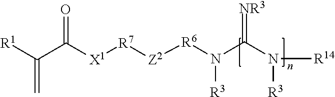

wherein:

R¹ is H or CH₃;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl;

R⁶ and R⁷ are each independently a (hetero)hydrocarbylene (e.g., a C₁-C₁₀ alkylene);

Z² is an ester, amide, urea, or urethane group, and

n is 1 or 2.

Such ligand monomers (M^{b}) may be made using condensation reactions as described in U.S. Pat. No. 8,846,203 (Bothof et al.).

In certain embodiments, the grafted ligand-functional polymer further includes a crosslinking monomer (M^{c}) (y is at least one), which has two or more free-radically polymerizable groups. In certain embodiments, the crosslinking monomer (M^{c}) is of the formula (XI):

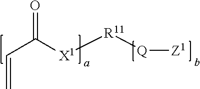

wherein:

Z¹ is an acryloyl or non-acryloyl, ethylenically unsaturated polymerizable group;

X¹ is —O— or —NR³—, where R³ is H or (hetero)hydrocarbyl;

Q is a divalent linking group selected from a covalent bond, —O—, —NR¹—, —CO₂—, and —C(O)NR¹—, where R¹ is H or CH₃;

R¹¹ is an alkylene group of valence a+b, and optionally containing one or more catenary oxygen atoms and/or one or more hydroxyl groups; and

a and b are each at least one.

In certain embodiments, the crosslinking monomer (M^{c}) includes a poly(alkylene oxide) compound having at least one acryloyl group and at least one additional ethylenically unsaturated, free-radically polymerizable group. In certain embodiments, the crosslinking monomer (M^{c}) is of the formula (XII):

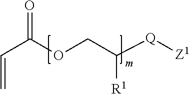

wherein:

Z¹ is an acryloyl or non-acryloyl, polymerizable ethylenically unsaturated group;

R¹ is H or CH₃;

m is from 2 to 100; and

Q is a divalent linking group selected from a covalent bond, —O—, —NR¹—, —CO₂— and —C(O)NR¹—, where R¹ is H or CH₃.

Examples of suitable crosslinking (M^{c}) monomers include di(meth)acrylates of poly(ethylene oxide) polymers and copolymers, such as poly(ethylene oxide-co-propylene oxide) copolymer, partially acrylated polyols, such as 3-(acryloxy)-2-hydroxypropylmethacrylate).

In certain embodiments, the grafted ligand-functional polymer further includes a hydrophilic monomer (M^{d}) (z is at least one), which has a free-radically polymerizable group and a hydrophilic group. The hydrophilic group may also include ionic groups that are positively charged, negatively charged, or neutral. In certain embodiments, the hydrophilic monomer (M^{d}) is a neutral monomer of the formula (XIII):

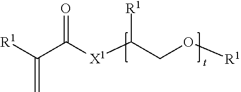

wherein:

each R¹ is independently H or CH₃;

X¹ is —O— or —NR³—, where R³ is H or (hetero)hydrocarbyl; and

t is from 2 to 100.

Examples of suitable hydrophilic (M^{d}) monomers include poly(alkylene oxide) monomers.

The anion exchange depth filters can be made using standard techniques, such as those described, for example, in U.S. Pat. No. 9,821,276 (Berrigan et al.) and U.S. Pat. No. 8,846,203 (Bothof et al.).

### Salt-Tolerant Anion Exchange Nonfibrous Porous Filter Element

Although the salt-tolerant anion exchange nonfibrous porous filter element is preferably a porous membrane, it is not a depth filter and its primary purpose is not to remove particulates. Exemplary salt-tolerant anion exchange nonfibrous porous filter elements include a nonfibrous base substrate and a grafted copolymer that includes interpolymerized cationic nitrogen-containing ligand monomers (i.e., ligands) as disclosed in U.S. Pat. No. 9,821,276 (Berrigan et al.), or grafted ligand-functional polymers as disclosed in U.S. Pat. No. 8,846,203 (Bothof et al.), and as used in the depth filters described above.

Salt-tolerant nitrogen-containing ligands typically include primary amines or guanidinyl groups, with guanidinyl-containing ligands being more salt tolerant than primary amines. Secondary amines, tertiary amines, and quaternary ammonium groups are typically not salt tolerant as defined herein. In certain embodiments, the salt-tolerant ligands are cationic nitrogen-containing ligand monomers that include a guanidinyl-containing ligand monomer of the formulas (III) and (IV) (described above), along with monomers of formula (XIV) (described above) prepared as described, for example, in U.S. Pat. No. 10,239,828 (Rasmussen et al.) and U.S. Pat. No. 8,846,203 (Bothof et al.).

The salt-tolerant anion exchange nonfibrous porous filter element is useful under conditions of high salt concentration or high ionic strength, i.e., they are “salt tolerant.” The term “salt” is meant to include all low molecular weight ionic species that contribute to the conductivity of the solution. Salt tolerance is important at least because many process solutions used in biopharmaceutical or enzyme manufacture have conductivities in the range of 15-30 mS/cm (approximately 150-300 mM salt) or more. Salt tolerance can be measured in comparison to that of the conventional quaternary amine or Quat ligand (e.g., trimethylammonium ligand), whose primarily electrostatic interactions with many biological species rapidly deteriorates at conductivities three- to six-fold less than the target range. For example, membranes derivatized with the conventional Quat ligand exhibit a drop in φX174 viral clearance from a six log-reduction value (LRV) to a one (1) LRV in going from 0 to 50 mM NaCl (ca. 5-6 mS/cm conductivity). Viruses, such as φX174, which have isoelectric points close to 7 (are neutral or near-neutral) are extremely difficult to remove from process streams. Similar problems are observed when attempting to remove other biological species from process fluids. For example, when attempting to remove positively charged proteins such as host cell proteins through the use of filtration devices functionalized with conventional Quat ligands, the process fluid may have to be diluted two-fold or more in order to reduce the conductivity to an acceptable range. This is expensive and dramatically increases the overall processing time.

Nonfibrous porous filter elements are advantageous for the salt-tolerant membranes over the fibrous porous substrates due to higher membrane surface area and higher graft densities of ligand-functional polymers that can be achieved, thus leading to higher capacities for removal of impurities. Also, the smaller pore sizes of the nonfibrous porous filter elements and the better control of the pore size distribution provides better control in separation of impurities, particularly biological molecules. Thus, nonfibrous porous filter elements may offer advantages over fibrous media in the salt-tolerant membranes in a flow-through purification process.

The nonfibrous porous filter element base substrate may be formed from any suitable thermoplastic polymeric material as described above for the depth filters.

In some embodiments, the porous base substrate is a microporous membrane such as a thermally-induced phase separation (TIPS) membrane. TIPS membranes are often prepared by forming a homogenous solution of a thermoplastic material and a second material above the melting point of the thermoplastic material. Upon cooling, the thermoplastic material crystallizes and phase separates from the second material. The crystallized thermoplastic material is often stretched. The second material is optionally removed either before or after stretching. Microporous membranes are further disclosed in U.S. Pat. No. 4,539,256 (Shipman), U.S. Pat. No. 4,726,989 (Mrozinski), U.S. Pat. No. 4,867,881 (Kinzer), U.S. Pat. No. 5,120,594 (Mrozinski), U.S. Pat. No. 5,260,360 (Mrozinski et al.), and U.S. Pat. No. 5,962,544 (Waller). Further, the microporous film can be prepared from ethylene-vinyl alcohol copolymers as described in U.S. Pat. No. 5,962,544 (Waller).

Some exemplary TIPS membrane comprise poly(vinylidene fluoride) (PVDF), polyolefins such as polyethylene homo- or copolymers or polypropylene homo- or copolymers, vinyl-containing polymers or copolymers such as ethylene-vinyl alcohol copolymers and butadiene-containing polymers or copolymers, and acrylate-containing polymers or copolymers. TIPS membranes comprising PVDF are further described in U.S. Pat. No. 7,338,692 (Smith et al.).

In another exemplary embodiment the porous bases substrate is a microporous membrane such as a solvent-induced phase separation (SIPS) membrane. SIPS membranes are often prepared by forming a homogenous solution of a thermoplastic material and a second material (a solvent), the solution is cast into a film or hollow fiber form, then immersed in a nonsolvent bath. The nonsolvent causes the thermoplastic material to solidify, or phase separate, and also extracts out the solvent, leaving a porous polymeric membrane. Examples of SIPS membranes prepared from polyamides include a nylon microporous film or sheet, such as those described in U.S. Pat. No. 6,056,529 (Meyering et al.), U.S. Pat. No. 6,267,916 (Meyering et al.), U.S. Pat. No. 6,413,070 (Meyering et al.), U.S. Pat. No. 6,776,940 (Meyering et al.), U.S. Pat. No. 3,876,738 (Marinacchio et al.), U.S. Pat. No. 3,928,517 (Knight et al.), U.S. Pat. No. 4,707,265 (Knight et al.), and U.S. Pat. No. 5,458,782 (Hou et al.). Other examples include microporous membranes prepared from polysulfones and polyethersulfones, many of which are commercially available from 3M Company, St. Paul, Minn., under the tradenames MicroPES and DuraPES.

The salt-tolerant membrane filters can be made using standard techniques, such as those described, for example, in U.S. Pat. No. 9,821,276 (Berrigan et al.), U.S. Pat. No. 8,846,203 (Bothof et al.), and U.S. Pat. No. 10,239,828 (Rasmussen et al.).

### Cation Exchange Nonfibrous Porous Filter Element

The cation exchange nonfibrous porous filter element includes a nonfibrous base substrate as disclosed above for the salt-tolerant anion exchange nonfibrous porous filter elements. It can be multimodal or mixed-mode. Multimodal or mixed-mode means that the filter element interacts with its target species by cation exchange and at least one other mode of interaction (e.g., hydrophobic interaction or hydrogen-bonding interaction).

In certain embodiments, the cation exchange nonfibrous porous filter element includes: a nonfibrous porous filter element; and disposed on the nonfibrous porous filter element, a polymer including: a hydrocarbon backbone and a plurality of pendant groups attached to the hydrocarbon backbone, wherein each of a first plurality of pendant groups includes: at least one acidic group or salt thereof; and a spacer group that directly links the at least one acidic group or salt thereof to the hydrocarbon backbone by a chain of at least 6 catenated atoms.

In certain embodiments, the spacer group includes a chain having at least 8 catenated atoms. In certain embodiments, the spacer group is a catenated heteroatom-containing hydrocarbon group. In certain embodiments, the spacer group includes at least one hydrogen bonding moiety, which is defined as a moiety including at least one hydrogen bond donor and at least one hydrogen bond acceptor (both of which are heteroatom-containing). In certain embodiments, the spacer group includes at least two hydrogen bonding moieties or includes at least one hydrogen bonding moiety and at least one hydrogen bond acceptor that is distinct from (not part of) the hydrogen bonding moiety. In certain embodiments, the spacer group includes at least two hydrogen bond donors, at least two hydrogen bond acceptors, or both.

In certain embodiments, the at least one acidic group or salt thereof of the polymer disposed on the nonfibrous porous filter element is present at a density of at least 0.01 (or at least 0.02) mmole/gram of cation exchange nonfibrous porous filter element. In certain embodiments, the at least one acidic group or salt thereof of the polymer disposed on the nonfibrous porous filter element is present at a density of up to 0.6 mmole/gram of cation exchange nonfibrous porous filter element.

In certain embodiments, the at least one acidic group or salt thereof is selected from a carboxy group, a phosphono group, a phosphato group, a sulfono group, a sulfato group, a boronato group, and a combination thereof.

In certain embodiments, the polymer includes interpolymerized units of at least one monomer including: at least one ethylenically unsaturated group; at least one acidic group or salt thereof; and a spacer group that directly links the at least one ethylenically unsaturated group and the at least one acidic group or salt thereof by a chain of at least 6 catenated atoms.

In certain embodiments, the at least one ethylenically unsaturated group is selected from an ethenyl group, a 1-alkylethenyl group, and a combination thereof.

In certain embodiments, the polymer is covalently attached to the nonfibrous porous filter element.

In certain embodiments, the polymer is a copolymer.

In certain embodiments, the copolymer includes a hydrocarbon backbone and a plurality of pendant groups attached to the hydrocarbon backbone, wherein: each of a first plurality of pendant groups includes: at least one acidic group or salt thereof; and a spacer group that directly links the at least one acidic group or salt thereof to the hydrocarbon backbone by a chain of at least 6 catenated atoms; and each of a second plurality of pendant groups includes: at least one acidic group or salt thereof; and a spacer group that directly links the at least one acidic group or salt thereof to the hydrocarbon backbone by a chain of at least 6 catenated atoms; wherein the first plurality of pendant groups are different than the second plurality of pendant groups; and wherein a mole ratio of the first plurality of pendant groups to the second plurality of pendant groups is in a range of 95:5 to 5:95.

In certain embodiments, the copolymer covalently attached to the nonfibrous porous filter element includes a reaction product of a monomer composition including: a first monomer including: at least one ethylenically unsaturated group; at least one acidic group or salt thereof; and a spacer group that directly links the at least one ethylenically unsaturated group and the at least one acidic group or salt thereof by a chain of at least 6 catenated atoms; and a second monomer including: at least one ethylenically unsaturated group; at least one acidic group or salt thereof; and a spacer group that directly links the at least one ethylenically unsaturated group and the at least one acidic group by a chain of at least 6 catenated atoms; wherein the second monomer is different than the first monomer; and wherein a mole ratio of the first monomer to the second monomer is in a range of 95:5 to 5:95.

In certain embodiments, the at least one ethylenically unsaturated group of the first monomer and/or second monomer is selected from an ethenyl group, a 1-alkylethenyl group, and a combination thereof. In certain embodiments, the first monomer is one of a class represented by the following general formula (XV):

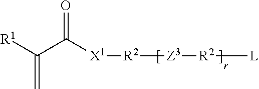

wherein:

R¹ is H or CH₃;

each R² is independently a (hetero)hydrocarbylene;

X¹ is —O— or —NR³—, where R³ is H or (hetero)hydrocarbyl;

Z³ is a heterohydrocarbylene group comprising at least one hydrogen bond donor, at least one hydrogen bond acceptor, or a combination thereof;

r is 0 or 1; and

L is a functional group comprising at least one acidic group or salt thereof.

In this context, a “hydrogen bond acceptor” means a heteroatom selected from oxygen, nitrogen, and sulfur that has a lone electron pair; and a “hydrogen bond donor” means a moiety consisting of a hydrogen atom covalently bonded to a heteroatom selected from oxygen, nitrogen, and sulfur. Hydrogen bond donors include, for example, donors such as imino, thiol, or hydroxy. Hydrogen bond acceptors include, for example, acceptors in the form of carbonyl, carbonyloxy, or ether oxygen.

In certain embodiments, the second monomer is one of a class represented by the general formula (XV) as well.

The cation exchange membrane filters can be made using standard techniques, such as those described, for example, in U.S. Publication No. 2019/0194250 (Colak Atan et al.) and International Publication No. WO 2018/048696 (Vail et al.).

### Devices

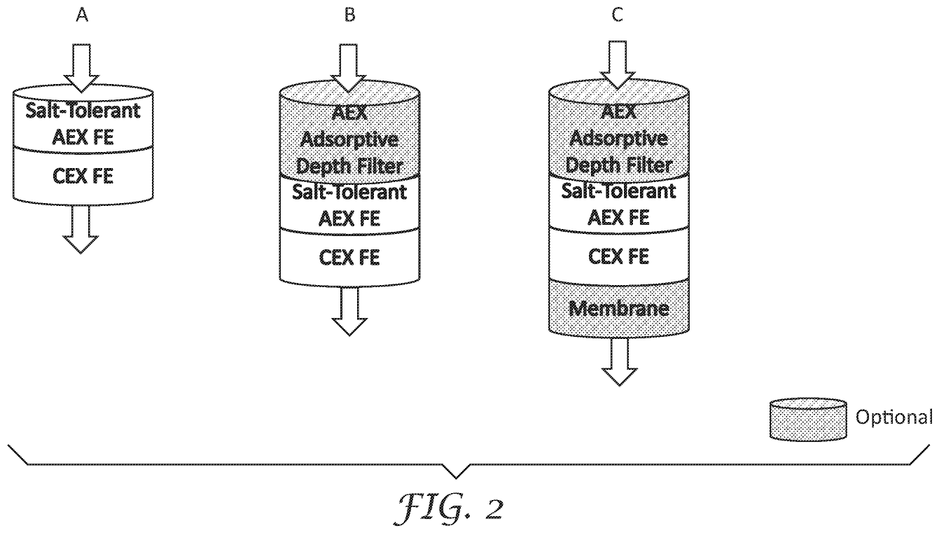

In a further embodiment, the present disclosure provides a filter cartridge that includes a salt-tolerant anion exchange nonfibrous porous filter element (Salt-Tolerant AEX FE) as described herein and a cation exchange nonfibrous porous filter element (CEX FE) as described herein, preferably positioned downstream from the salt-tolerant filter element, as shown in FIG. 2A. The devices may also include an anion exchange adsorptive depth filter (AEX Adsorptive Depth Filter), preferably positioned upstream of the salt-tolerant filter element, as shown in FIG. 2B. The relative orientations of these filter elements may be as described herein for the processes. The device may include other conventional filter elements, such as one or more microporous membranes, as shown in FIG. 2C.

### Exemplary Embodiments

Embodiment 1 is a flow-through process for purifying a target molecule from a biological solution in a sample that includes the target molecule, the process comprising:

optionally, contacting the sample with an anion exchange adsorptive depth filter;

optionally, conducting a buffer exchange with the sample before and/or after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element;

wherein the flow-through process comprises one or two buffer exchanges, with no buffer exchange between the sample contacting the salt-tolerant anion exchange nonfibrous porous filter element and the cation exchange nonfibrous porous filter element.

Embodiment 2 is the flow-through process of embodiment 1, comprising:

contacting the sample with an anion exchange adsorptive depth filter;

optionally, conducting a buffer exchange with the sample before and/or after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 3 is the flow-through process of embodiment 2, comprising:

contacting the sample with an anion exchange adsorptive depth filter;

conducting a buffer exchange with the sample before and/or after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 4 is the flow-through process of embodiment 3, comprising:

contacting the sample with an anion exchange adsorptive depth filter;

conducting a buffer exchange with the sample after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 5 is the flow-through process of embodiment 3, comprising:

contacting the sample with an anion exchange adsorptive depth filter;

conducting a buffer exchange with the sample before contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 6 is the flow-through process of embodiment 3, comprising:

contacting the sample with an anion exchange adsorptive depth filter;

conducting a buffer exchange with the sample before and after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 7 is the flow-through process of any previous embodiment, comprising conducting a buffer exchange with the sample after contacting the sample with a cation exchange nonfibrous porous filter element.

Embodiment 8 is a flow-through process for purifying a target molecule from a biological solution in a sample that includes the target molecule, the process comprising:

contacting the sample with an anion exchange adsorptive depth filter;

immediately thereafter, contacting the sample with a salt-tolerant anion exchange nonfibrous porous filter element; and
immediately thereafter, contacting the sample with a cation exchange nonfibrous porous filter element;
wherein the flow-through process comprises no buffer exchanges.

Embodiment 9 is a flow-through process for purifying a target molecule from a biological solution in a sample that includes the target molecule, the process comprising:

contacting the sample with an anion exchange adsorptive depth filter;

conducting a buffer exchange with the sample after contacting the sample with an anion exchange adsorptive depth filter;

contacting the sample with a salt-tolerant anion exchange nonfibrous porous filter element; and

immediately thereafter, contacting the sample with a cation exchange nonfibrous porous filter element.

Embodiment 10 is a flow-through process for purifying a target molecule from a biological solution in a sample that includes the target molecule, the process comprising:

conducting a buffer exchange with the sample;

contacting the sample with a salt-tolerant anion exchange nonfibrous porous filter element; and

immediately thereafter, contacting the sample with a cation exchange nonfibrous porous filter element.

Embodiment 11 is the flow-through process of any previous embodiment wherein the target molecule comprises a monoclonal antibody.

Embodiment 12 is the flow-through process of any previous embodiment wherein the biological solution comprises a neutralized viral inactivation pool.

Embodiment 13 is the flow-through process of any of embodiments 2 through 9, and 11-12 as dependent on one of embodiments 2 through 9, wherein the anion exchange adsorptive depth filter comprises a porous substrate comprising immobilized anion exchange ligands.

Embodiment 14 is the flow-through process of embodiment 13 wherein the anion exchange ligands of the anion exchange adsorptive depth filter comprise cationic nitrogen-containing ligands.

Embodiment 15 is the flow-through process of embodiment 14 wherein the cationic nitrogen-containing ligands of the anion exchange adsorptive depth filter comprise a primary amine, a secondary amine, a tertiary amine, or combinations thereof.

Embodiment 16 is the flow-through process of embodiment 15 wherein the cationic nitrogen-containing ligands of the anion exchange adsorptive depth filter comprise a quaternary ammonium-containing ligand, a guanidinyl-containing ligand, or a combination thereof.

Embodiment 17 is the flow-through process of any of embodiments 14 through 16 wherein the anion exchange adsorptive depth filter comprises a copolymer grafted article comprising a porous substrate and a copolymer grafted thereto, wherein the grafted copolymer comprises interpolymerized monomer units comprising cationic nitrogen-containing ligands.

Embodiment 18 is the flow-through process of embodiment 17 wherein the grafted copolymer of the anion exchange adsorptive depth filter comprises interpolymerized monomer units comprising:

a cationic nitrogen-containing ligand monomer selected from the group of quaternary ammonium-containing ligand monomers, guanidinyl-containing ligand monomers, and combinations thereof;

an amide monomer;

an oxy monomer selected from the group of epoxy functional monomer units, alkyl ether functional monomer units, and combinations thereof; and

a poly(alkylene oxide) monomer.

Embodiment 19 is the flow-through process of embodiment 18 wherein the grafted copolymer of the anion exchange adsorptive depth filter comprises interpolymerized monomer units comprising:

10 to 50 parts by weight of a cationic nitrogen-containing ligand monomer, wherein the cationic nitrogen-containing ligand monomer is selected from the group of quaternary ammonium-containing ligand monomers, guanidinyl-containing ligand monomers, and combinations thereof;

10 to 80 parts by weight of an amide monomer;

10 to 40 parts by weight of an oxy monomer selected from the group of epoxy functional monomer units, alkyl ether functional monomer units, and combinations thereof; and

0 to 30 parts by weight of a poly(alkylene oxide) monomer;

wherein the sum of the monomers is 100 parts by weight.

Embodiment 20 is the flow-through process of embodiment 18 or 19 wherein the cationic nitrogen-containing ligand monomer used to make the anion exchange adsorptive depth filter is of the formula (I):

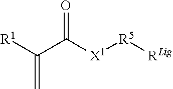

wherein:

R¹ is H or CH₃;

X¹ is —O— or —NR³—, where R³ is H or (hetero)hydrocarbyl;

R⁵ is a (hetero)hydrocarbylene; and

R^{Lig} is a quaternary ammonium ligand group or a guanidinyl-containing ligand group.

Embodiment 21 is the flow-through process of embodiment 20 wherein the cationic nitrogen-containing ligand monomer used to make the anion exchange adsorptive depth filter is a quaternary ammonium monomer of the formula (II):

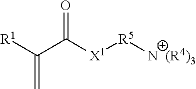

wherein:

R¹ is H or CH₃;

X¹ is —O— or —NR³—, where R³ is H or (hetero)hydrocarbyl;

R⁵ is a (hetero)hydrocarbylene; and

each R⁴ is independently alkyl or aryl.

Embodiment 22 is the flow-through process of embodiment 20 wherein the cationic nitrogen-containing ligand monomer used to make the anion exchange adsorptive depth filter is a guanidinyl-containing ligand monomer is of the formula (III) or (IV):

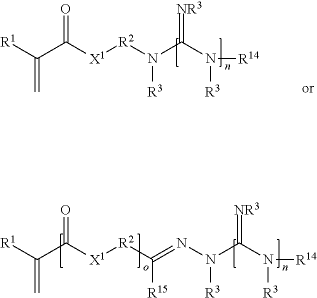

wherein:

R¹ is H or CH₃;

R² is a (hetero)hydrocarbylene (e.g., having 1 to 20 carbon atoms);

each R³ is independently H or (hetero)hydrocarbyl;

R¹⁴ is H, (hetero)hydrocarbyl, or —N(R³)₂, where each R³ is independently H or (hetero)hydrocarbyl;

R¹⁵ is H or hydrocarbyl (e.g., a C₁-C₄ alkyl group, or an aryl group);

X¹ is —O— or —NR³—, where R³ is H or (hetero)hydrocarbyl;

o is 0 or 1; and

n is 1 or 2.

Embodiment 23 is the flow-through process of any of embodiments 18 through 22 wherein the amide monomer used to make the anion exchange adsorptive depth filter is of the formula (V) or (VI):

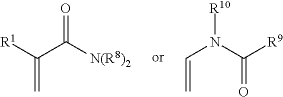

wherein:

R¹ is H or CH₃;

each R⁸ is independently hydrogen, alkyl, or aryl; and

R⁹ and R¹⁰ are alkyl groups, or may be taken together to form a 5 or 6-membered ring.

Embodiment 24 is the flow-through process of any of embodiments 18 through 23 wherein the oxy monomer used to make the anion exchange adsorptive depth filter is of the formula (VII):

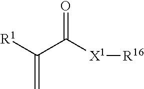

wherein:

R¹ is H or CH₃;

X¹ is —O— or —NR³—, where R³ is H or (hetero)hydrocarbyl; and

R¹⁶ is an epoxy-functional or ether-functional hydrocarbyl group.

Embodiment 25 is the flow-through process of embodiment 24 wherein the oxy monomer used to make the anion exchange adsorptive depth filter is of the formula (VIII):

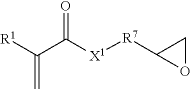

wherein:

R¹ is H or CH₃;

X¹ is —O— or —NR³—, where R³ is H or (hetero)hydrocarbyl; and

R⁷ is a (hetero)hydrocarbylene (e.g., a C₁-C₆ alkylene).

Embodiment 26 is the flow-through process of embodiment 13 wherein the anion exchange adsorptive depth filter comprises a porous substrate and a ligand-functional polymer grafted thereto, wherein the grafted ligand-functional polymer is of the formula:

-(M^{PI})_{w}-(M^{b})ₓ-(M^{c})_{y}-(M^{d})_{z}

wherein:

-(M^{PI})_{w} represent the residue of grafted photoinitiator monomers, where w is zero or at least one;

-(M^{b})ₓ represents polymerized ligand monomers, having “x” polymerized monomer units, where x is at least one;

-(M^{c})_{y} represents polymerized crosslinking monomers, having y polymerized monomer units, where y may be zero or at least one; and

-(M^{d})_{z} represents polymerized hydrophilic monomers, having z polymerized monomer units, where z may be zero or at least one.

Embodiment 27 is the flow-through process of embodiment 26 wherein the ligand monomer (M^{b}) used to make the anion exchange adsorptive depth filter is of formula (X):

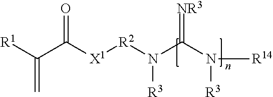

wherein:

R¹ is H or CH₃;

R² is a (hetero)hydrocarbylene;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl, and

n is 1 or 2.

Embodiment 28 is the flow-through process of embodiment 27 wherein the ligand monomer (M^{b}) used to make the anion exchange adsorptive depth filter is of the formula (XIV):

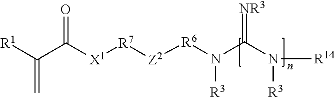

wherein:

R¹ is H or CH₃;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl;

R⁶ and R⁷ are each independently a (hetero)hydrocarbylene (e.g., a C₁-C₁₀ alkylene);

Z² is an ester, amide, urea, or urethane group, and

n is 1 or 2.

Embodiment 29 is the flow-through process of any of embodiments 26 through 28 wherein the grafted ligand-functional polymer of the anion exchange adsorptive depth filter further comprises a crosslinking monomer (M^{c}) (y is at least one), which has two or more free-radically polymerizable groups.

Embodiment 30 is the flow-through process of embodiment 29 wherein the crosslinking monomer (M^{c}) used to make the anion exchange adsorptive depth filter is of the formula (XI):

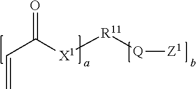

wherein:

Z¹ is an acryloyl or non-acryloyl, ethylenically unsaturated polymerizable group;

X¹ is —O— or —NR³—, where R³ is H or (hetero)hydrocarbyl;

Q is a divalent linking group selected from a covalent bond, —O—, —NR¹—, —CO₂—, and —C(O)NR¹—, where R¹ is H or CH₃;

R¹¹ is an alkylene group of valence a+b, and optionally containing one or more catenary oxygen atoms and/or one or more hydroxyl groups; and

a and b are each at least one.

Embodiment 31 is the flow-through process of embodiment 29 wherein the crosslinking monomer (M^{c}) used to make the anion exchange adsorptive depth filter is of the formula (XII):

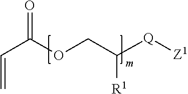

wherein:

Z¹ is an acryloyl or non-acryloyl, polymerizable ethylenically unsaturated group;

R¹ is H or CH₃;

m is from 2 to 100; and

Q is a divalent linking group selected from a covalent bond, —O—, —NR¹—, —CO₂— and —C(O)NR¹—, where R¹ is H or CH₃.

Embodiment 32 is the flow-through process of any of embodiments 26 through 31 wherein the grafted ligand-functional polymer of the anion exchange adsorptive depth filter further comprises a hydrophilic monomer (M^{d}) (z is at least one), which has a free-radically polymerizable group and a hydrophilic group.

Embodiment 33 is the flow-through process of embodiment 32 wherein the hydrophilic monomer (M^{d}) used to make the anion exchange adsorptive depth filter is of the formula (XIII):

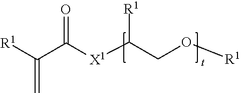

wherein:

each R¹ is independently H or CH₃;

X¹ is —O— or —NR³—, where R³ is H or (hetero)hydrocarbyl; and

t is from 2 to 100.

Embodiment 34 is the flow-through process of any previous embodiment wherein the salt-tolerant anion exchange nonfibrous porous filter element comprises a nonfibrous porous filter element comprising immobilized anion exchange ligands.

Embodiment 35 is the flow-through process of embodiment 34 wherein the anion exchange ligands of the salt-tolerant anion exchange nonfibrous porous filter element comprise cationic nitrogen-containing ligands.

Embodiment 36 is the flow-through process of embodiment 35 wherein the cationic nitrogen-containing ligands of the salt-tolerant anion exchange nonfibrous porous filter element comprises a guanidinyl-containing ligand.

Embodiment 37 is the flow-through process of embodiment 35 or 36 wherein the salt-tolerant anion exchange nonfibrous porous filter element comprises a copolymer grafted article comprising a nonfibrous porous filter element and a copolymer grafted thereto, wherein the grafted copolymer comprises interpolymerized monomer units comprising cationic nitrogen-containing ligands.

Embodiment 38 is the flow-through process of embodiment 37 wherein the grafted copolymer of the salt-tolerant anion exchange nonfibrous porous filter element comprises interpolymerized monomer units comprising:

a guanidinyl-containing ligand monomer;

an amide monomer;

an oxy monomer selected from the group of epoxy functional monomer units, alkyl ether functional monomer units, and combinations thereof; and

a poly(alkylene oxide) monomer.

Embodiment 39 is the flow-through process of embodiment 38 wherein the grafted copolymer of the salt-tolerant anion exchange nonfibrous porous filter element comprises interpolymerized monomer units comprising:

10 to 50 parts by weight of a guanidinyl-containing ligand monomers;

10 to 80 parts by weight of an amide monomer;

10 to 40 parts by weight of an oxy monomer selected from the group of epoxy functional monomer units, alkyl ether functional monomer units, and combinations thereof; and

0 to 30 parts by weight of a poly(alkylene oxide) monomer;

wherein the sum of the monomers is 100 parts by weight.

Embodiment 40 is the flow-through process of embodiment 38 or 39 wherein the cationic nitrogen-containing ligand monomer used to make the salt-tolerant anion exchange nonfibrous porous filter element is of the formula (I):

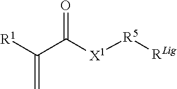

wherein:

R¹ is H or CH₃;

X¹ is —O— or —NR³—, where R³ is H or (hetero)hydrocarbyl;

R⁵ is a (hetero)hydrocarbylene; and

R^{Lig} is a guanidinyl-containing ligand group.

Embodiment 41 is the flow-through process of embodiment 40 wherein the cationic nitrogen-containing ligand monomer used to make the salt-tolerant anion exchange nonfibrous porous filter element is a guanidinyl-containing ligand monomer of the formula (III) or (IV):

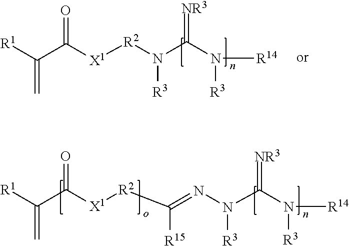

wherein:

R¹ is H or CH₃;

R² is a (hetero)hydrocarbylene (e.g., having 1 to 20 carbon atoms);

each R³ is independently H or (hetero)hydrocarbyl;

R¹⁴ is H, (hetero)hydrocarbyl, or —N(R³)₂, where each R³ is independently H or (hetero)hydrocarbyl;

R¹⁵ is H or hydrocarbyl (e.g., a C₁-C₄ alkyl group, or an aryl group);

X¹ is —O— or —NR³—, where R³ is H or (hetero)hydrocarbyl;

o is 0 or 1; and

n is 1 or 2.

Embodiment 42 is the flow-through process of embodiment 34 wherein the salt-tolerant anion exchange nonfibrous porous filter element comprises a nonfibrous porous filter element and a ligand-functional polymer grafted thereto, wherein the grafted ligand-functional polymer is of the formula:

-(M^{PI})_{w}-(M^{b})ₓ-(M^{c})_{y}-(M^{d})_{z}

wherein:

-(M^{PI})_{w} represent the residue of grafted photoinitiator monomers, where w is zero or at least one;

-(M^{b})ₓ represents polymerized ligand monomers, having “x” polymerized monomer units, where x is at least one;

-(M^{c})_{y} represents polymerized crosslinking monomers, having y polymerized monomer units, where y may be zero or at least one; and

-(M^{d})_{z} represents polymerized hydrophilic monomers, having z polymerized monomer units, where z may be zero or at least one.

Embodiment 43 is the flow-through process of embodiment 42 wherein the ligand monomer (M^{b}) used to make the salt-tolerant anion exchange nonfibrous porous filter element is of formula (X):

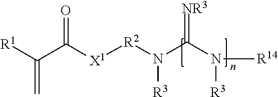

wherein:

R¹ is H or CH₃;

R² is a (hetero)hydrocarbylene;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl, and

n is 1 or 2.

Embodiment 44 is the flow-through process of embodiment 43 wherein the ligand monomer (M^{b}) used to make the salt-tolerant anion exchange nonfibrous porous filter element is of the formula (XIV):

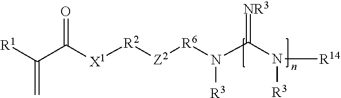

wherein:

R¹ is H or CH₃;

each R³ is independently H or (hetero)hydrocarbyl;

R¹⁴ is H, (hetero)hydrocarbyl, or —N(R³)₂, where each R³ is independently H or (hetero)hydrocarbyl;

X¹ is —O— or —NR³—, where R³ is H or (hetero)hydrocarbyl;

R⁶ and R⁷ are each independently a (hetero)hydrocarbylene (e.g., a C₁-C₁₀ alkylene); and

Z² is an ester, amide, urea, or urethane group, and

n is 1 or 2.

Embodiment 45 is the flow-through process of any of embodiments 42 through 44 wherein the grafted ligand-functional polymer of the salt-tolerant anion exchange nonfibrous porous filter element further comprises a crosslinking monomer (M^{c}) (y is at least one), which has two or more free-radically polymerizable groups.

Embodiment 46 is the flow-through process of embodiment 45 wherein the crosslinking monomer (M^{c}) used to make the salt-tolerant anion exchange nonfibrous porous filter element is of the formula (XI):

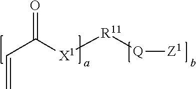

wherein:

Z¹ is an acryloyl or non-acryloyl, ethylenically unsaturated polymerizable group;

X¹ is —O— or —NR³—, where R³ is H or (hetero)hydrocarbyl;

Q is a divalent linking group selected from a covalent bond, —O—, —NR¹—, —CO₂—, and —C(O)NR¹—, where R¹ is H or CH₃;

R¹¹ is an alkylene group of valence a+b, and optionally containing one or more catenary oxygen atoms and/or one or more hydroxyl groups; and

a and b are each at least one.

Embodiment 47 is the flow-through process of embodiment 46 wherein the crosslinking monomer (M^{c}) used to make the salt-tolerant anion exchange nonfibrous porous filter element is of the formula (XII):

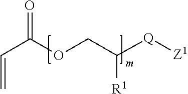

wherein:

Z¹ is an acryloyl or non-acryloyl, polymerizable ethylenically unsaturated group;

R¹ is H or CH₃;

m is from 2 to 100; and

Q is a divalent linking group selected from a covalent bond, —O—, —NR¹—, —CO₂— and —C(O)NR¹—, where R¹ is H or CH₃.

Embodiment 48 is the flow-through process of any of embodiments 42 through 47 wherein the grafted ligand-functional polymer of the salt-tolerant anion exchange nonfibrous porous filter element further comprises a hydrophilic monomer (M^{d}) (z is at least one), which has a free-radically polymerizable group and a hydrophilic group.

Embodiment 49 is the flow-through process of embodiment 48 wherein the hydrophilic monomer (M^{d}) used to make the salt-tolerant anion exchange nonfibrous porous filter element is of the formula (XIII):

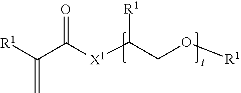

wherein:

each R¹ is independently H or CH₃;

X¹ is —O— or —NR³—, where R³ is H or (hetero)hydrocarbyl; and

t is from 2 to 100.

Embodiment 50 is the flow-through process of any previous embodiment wherein the cation exchange nonfibrous porous filter element comprises:

a nonfibrous porous filter element; and

disposed on the nonfibrous porous filter element, a polymer comprising:
a hydrocarbon backbone and a plurality of pendant groups attached to the hydrocarbon backbone, wherein each of a first plurality of pendant groups comprises:
   at least one acidic group or salt thereof; and
   a spacer group that directly links the at least one acidic group or salt thereof to the hydrocarbon backbone by a chain of at least 6 catenated atoms.

Embodiment 51 is the flow-through process of embodiment 50 wherein the chain of the spacer group has at least 8 catenated atoms.

Embodiment 52 is the flow-through process of embodiment 50 or 51 wherein the spacer group is a catenated heteroatom-containing hydrocarbon group.

Embodiment 53 is the flow-through process of any of embodiments 50 through 52 wherein the spacer group comprises at least one hydrogen bonding moiety.

Embodiment 54 is the flow-through process of any of embodiments 50 through 53 wherein the at least one acidic group or salt thereof of the polymer disposed on the nonfibrous porous filter element is present at a density of at least 0.01 (or at least 0.02) mmole/gram of cation exchange nonfibrous porous filter element.

Embodiment 55 is the flow-through process of any of embodiments 50 through 54 wherein the at least one acidic group or salt thereof of the polymer disposed on the nonfibrous porous filter element is present at a density of up to 0.6 mmole/gram of cation exchange nonfibrous porous filter element.

Embodiment 56 is the flow-through process of any of embodiments 50 through 55 wherein the at least one acidic group or salt thereof is selected from a carboxy group, a phosphono group, a phosphato group, a sulfono group, a sulfato group, a boronato group, and a combination thereof.

Embodiment 57 is the flow-through process of any of embodiments 50 through 56 wherein the polymer comprises interpolymerized units of at least one monomer comprising: at least one ethylenically unsaturated group; at least one acidic group or salt thereof; and a spacer group that directly links the at least one ethylenically unsaturated group and the at least one acidic group or salt thereof by a chain of at least 6 catenated atoms.

Embodiment 58 is the flow-through process of embodiment 57 wherein the at least one ethylenically unsaturated group is selected from an ethenyl group, a 1-alkylethenyl group, and a combination thereof.

Embodiment 59 is the flow-through process of any of embodiments 50 through 58 wherein the polymer is covalently attached to the nonfibrous porous filter element.

Embodiment 60 is the flow-through process of any of embodiments 50 through 59 wherein the polymer is a copolymer.

Embodiment 61 is the flow-through process of embodiment 60 wherein the copolymer comprises a hydrocarbon backbone and a plurality of pendant groups attached to the hydrocarbon backbone, wherein:
each of a first plurality of pendant groups comprises:
   at least one acidic group or salt thereof; and
   a spacer group that directly links the at least one acidic group or salt thereof to the hydrocarbon backbone by a chain of at least 6 catenated atoms; and
each of a second plurality of pendant groups comprises:
   at least one acidic group or salt thereof; and
   a spacer group that directly links the at least one acidic group or salt thereof to the hydrocarbon backbone by a chain of at least 6 catenated atoms;
wherein the first plurality of pendant groups are different than the second plurality of pendant groups; and
wherein a mole ratio of the first plurality of pendant groups to the second plurality of pendant groups is in a range of 95:5 to 5:95.

Embodiment 62 is the flow-through process of embodiment 61 wherein the copolymer covalently attached to the nonfibrous porous filter element comprises a reaction product of a monomer composition comprising:
a first monomer comprising:
   at least one ethylenically unsaturated group;
   at least one acidic group or salt thereof; and
   a spacer group that directly links the at least one ethylenically unsaturated group and the at least one acidic group or salt thereof by a chain of at least 6 catenated atoms; and
a second monomer comprising:
   at least one ethylenically unsaturated group;
   at least one acidic group or salt thereof; and
   a spacer group that directly links the at least one ethylenically unsaturated group and the at least one acidic group by a chain of at least 6 catenated atoms;
wherein the second monomer is different than the first monomer; and
wherein a mole ratio of the first monomer to the second monomer is in a range of 95:5 to 5:95.

Embodiment 63 is the flow-through process of embodiment 62 wherein the at least one ethylenically unsaturated group of the first monomer and/or second monomer is selected from an ethenyl group, a 1-alkylethenyl group, and a combination thereof.

Embodiment 64 is the flow-through process of embodiment 62 or 63 wherein the first monomer is one of a class represented by the following general formula (XV):

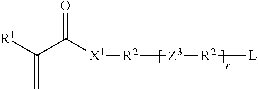

wherein:

R¹ is H or CH₃;

each R² is independently a (hetero)hydrocarbylene;

X¹ is —O— or —NR³—, where R³ is H or (hetero)hydrocarbyl;

Z³ is a (hetero)hydrocarbylene group comprising at least one hydrogen bond donor, at least one hydrogen bond acceptor, or a combination thereof;

r is 0 or 1; and

L is a functional group comprising at least one acidic group or salt thereof

Embodiment 65 is the flow-through process of any of embodiments 62 through 64 wherein the second monomer is one of a class represented by the following general formula (XV):

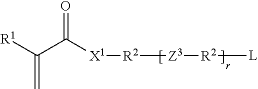

wherein:

R¹ is H or CH₃;

each R² is independently a (hetero)hydrocarbylene;

X¹ is —O— or —NR³—, where R³ is H or (hetero)hydrocarbyl;

Z³ is a (hetero)hydrocarbylene group comprising at least one hydrogen bond donor, at least one hydrogen bond acceptor, or a combination thereof;

r is 0 or 1; and

L is a functional group comprising at least one acidic group or salt thereof.

Embodiment 66 is a filter cartridge comprising a salt-tolerant anion exchange nonfibrous porous filter element as described herein and a cation exchange nonfibrous porous filter element as described herein.

Embodiment 67 is the filter cartridge of embodiment 66 wherein the cation exchange nonfibrous porous filter element is positioned downstream from the salt-tolerant anion exchange nonfibrous porous filter element.

Embodiment 68 is the filter cartridge of embodiment 66 or 67 wherein the salt-tolerant anion exchange nonfibrous porous filter element comprises a nonfibrous porous filter element comprising immobilized cationic nitrogen-containing ligands.

Embodiment 69 is the filter cartridge of any of embodiments 66 through 68 wherein the salt-tolerant anion exchange nonfibrous membrane comprises a nonfibrous porous filter element and a ligand-functional polymer grafted thereto, wherein the grafted ligand-functional polymer is of the formula:

-(M^{PI})_{w}-(M^{b})ₓ-(M^{c})_{y}-(M^{d})_{z},

wherein:
-(M^{PI})_{w} represent the residue of grafted photoinitiator monomers, where w is zero or at least one;
-(M^{b})ₓ represents polymerized ligand monomers, having “x” polymerized monomer units, where x is at least one;
-(M^{c})_{y} represents polymerized crosslinking monomers, having y polymerized monomer units, where y may be zero or at least one; and
-(M^{d})_{z} represents polymerized hydrophilic monomers, having z polymerized monomer units, where z may be zero or at least one.

Embodiment 70 is the filter cartridge of any of embodiments 66 through 69 wherein the cation exchange nonfibrous porous filter element comprises:

a nonfibrous porous filter element; and

disposed on the nonfibrous porous filter element, a polymer comprising:
a hydrocarbon backbone and a plurality of pendant groups attached to the hydrocarbon backbone, wherein each of a first plurality of pendant groups comprises:
   at least one acidic group or salt thereof; and
   a spacer group that directly links the at least one acidic group or salt thereof to the hydrocarbon backbone by a chain of at least 6 catenated atoms.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

Unless otherwise noted, all parts, percentages, ratios, etc. in the examples and the rest of the specification are by weight, and all reagents used in the examples were obtained, or are available, from general chemical suppliers such as, for example, Sigma-Aldrich Company, Saint Louis, Mo., or may be synthesized by conventional methods.

TABLE 1
Materials

| Description (abbreviation) | Source |
|---|---|
| SARTOBIND STIC PA, salt tolerant | Sartorius AG, Goettingen, |
| anion exchanger 96-well plate | Germany |
| SARTOBIND S, strong acidic cation | Sartorius AG |
| exchanger 96-well plate | |
| L-O-Phosphoserine | TCI America, Portland, OR |
| O-Phosphorylethanolamine | TCI America |
| Sodium phosphate monobasic | Sigma-Aldrich Corporation, St. |
| | Louis, MO |
| Sodium phosphate dibasic | Sigma-Aldrich Corporation |
| Sodium chloride (NaCl) | EMD Chemicals, Gibbstown, NJ |
| Tris(hydroxymethyl)- | Alfa Aesar, Ward Hill, MA |
| aminomethane (Tris) | |
| 2-Isocyanatoethyl methacrylate (IEM) | Showa Denko KK, Kanagawa, |
| | Japan |
| 4,4-dimethyl-2-vinyl-4H-oxazol-5- | SNPE, Inc, Princeton, NJ |
| one (vinyldimethylazlactone, | |
| VDM) (CAS# 29513-26-6) | |
| 4-(3-Sulfopropyloxy)benzophenone, | Described in Japanese Patent |
| sodium salt (SBP) | No. 47040913 (Teijin Ltd.) |

### Buffer Changes or Adjustments for Monoclonal Antibody-Containing Solutions

Conductivity adjustments were performed using 4 Molar (4 M) NaCl. Adjustment of pH was accomplished using acetic acid (200 millimolar (mM) or 500 mM) and Tris base (2 M). The conductivity measurements were determined using an Accumet Excel XL50 conductivity meter (Fisher Scientific, Hampton, N.H.). The pH measurements were determined using a VWR SYMPHONY benchtop pH meter (VWR International, Radnor, Pa.).

### Filter Element Challenge Plates

The finished filter elements (FE-A-FE-L) were cut into 7.5 millimeter (mm) diameter disks. For the anion exchange filter element (FE-L), a single disk was loaded into each well of a 96-well EMPORE Filter Plate (Model 6065, 3M Corporation, St. Paul, Minn.). For each type of cation exchange filter element, two disks of the same filter element were loaded into each well of a 96-well EMPORE Filter Plate. The filter elements were held in place with a plastic O-ring. Total working filter volume of each well was about 8.6 microliters. Each challenge plate loaded with a filter element was placed onto a 96-well deep well collection plate (Thermo Fisher Scientific, Waltham, Mass.) prior to centrifugation for sample collection. Centrifugation was performed using an Allegra 25R centrifuge (Beckman Coulter, Brea, Calif.). Commercial challenge plates (Sartorius SARTOBIND 96-well plates) were received from the manufacturer and used according to the manufacturer's instructions.

### Target Molecule and Impurity Analysis Methods

Chinese hamster ovary (CHO) host cell protein (HCP) concentration was determined using a CHO HCP ELISA Kit, 3G (Cygnus Technologies, Southport, N.C.) according to the manufacturer's protocol.

Protein concentrations of the mAb were determined by Beer's law using the absorbance at 280 nanometers and an extinction coefficient of 1.36. A SpectraMax M5 spectrophotometer (Molecular Devices, San Jose, Calif.) was used to determine the absorbance.

Monomer yield (‘% monomeric mAb yield’) and high molecular weight (HMW) species composition of filtered solutions were analyzed by size exclusion chromatography (SEC) using a Shimadzu Prominence HPLC System (Shimadzu Scientific Instruments, Columbia, Md.) with a TOSOH TSKgel G3000SW_{XL} column (Tosoh BioScience LLC, Griesheim, Germany) (analysis conditions: injection volume of 20 microliters; flow rate of 1 mL/minute; mobile phase: 100 mM sodium phosphate, 300 mM NaCl, pH 6.9; detection at 280 nanometers). Monomer yield and high molecular weight (HMW) species composition were determined by comparison of peak areas of the starting and filtered solutions and the results were reported as the average of two replicates.

The percent of HMW species present in the mAb solution was determined using the SEC chromatography method described above. The peak area of the HMW species component (Peak A) of the sample was compared to the sum of the sample peak areas (Total Peak Area). The “% HMW Composition” was calculated according to Equation 1.

The percent removal of HMW species was determined using the SEC chromatography method described above and measuring the peak area for the aggregated component before performing the separation process (Peak B) and after performing a separation process using a depth filter or filter element challenge plates described in the examples (Peak C). The “% HMW Removed” was calculated according to Equation 2.

The percent yield of monomeric monoclonal antibody (mAb) was determined using the SEC chromatography method described above and measuring the peak area for the monomeric component before performing the separation process (Peak D) and after performing a separation process by using a depth filter or filter element challenge plates described in the examples (Peak E). The “% Monomeric mAb Yield” was calculated according to Equation 3.

The percent removal of HCP was determined using the HCP quantification method described above and measuring the HCP concentration before performing the separation process ([HCP before]) and after performing the separation process ([HCP after])). The “% HCP Removed” was calculated according to Equation 4.

$$\% HMW Composition = ( \frac{Peak Area A}{Total Peak Area} ) x 100 \qquad Equation 1$$

$$\% HMW Removed = 100 - ( \frac{Peak Area C}{Peak Area B} ) x 100 \qquad Equation 2$$

$$\% Monomeric mAb Yield = 100 - ( \frac{Peak Area E}{Peak Area D} ) x 100 \qquad Equation 3$$

$$\% HCP Removed = 100 - ( \frac{[ HCP after ]}{[ HCP before ]} ) x 100 \qquad Equation 4$$

### Preparation of Cation Exchange Nonfibrous Porous Filter Element A (FE-A)

A coating solution was prepared by mixing 4-aminobutyric acid sodium salt/IEM monomer solution (IEM-GABA) (prepared as described in Monomer Example B of International Publication No. WO 2018/048698) (Vail et al.) (6.73 grams (g) of a 20.8% w/w solution in deionized water) with sulfonated benzophenone (S-BP) (250 microliters of a 0.1 gram per milliliter (g/mL) solution in deionized water). Deionized water (13.02 g) was added to provide a mixture approximately 0.25 M in monomer. A nylon membrane substrate (18 centimeter (cm)×23 cm; nylon 66 membrane, single reinforced layer nylon three-zone membrane, nominal pore size 1.8 micrometer (um or micron), #080ZN, obtained from 3M Purification, Inc., Meriden, Conn.) was placed on a sheet of polyester film and the coating solution was pipetted onto the top surface of the substrate. The coating solution was allowed to soak into the substrate for about 1 minute, and then a second sheet of polyester film was placed on top of the substrate. A 2.28 kilogram (kg) cylindrical weight was rolled over the top of the resulting three-layer sandwich to squeeze out excess coating solution. Ultraviolet (UV)-initiated grafting was conducted by irradiating the sandwich using a UV stand (Classic Manufacturing, Inc., Oakdale, Minn.) equipped with 18 bulbs [Sylvania RG2 40W F40/350BL/ECO, 10 above and 8 below the substrate, 1.17 meters (46 inches) long, spaced 5.1 cm (2 inches) on center], with an irradiation time of 15 minutes. The polyester sheets were removed, and the resulting functionalized substrate was placed in a 1000 mL polyethylene bottle. The bottle was filled with 0.9% (w/w) saline, sealed, and placed on a roller for 30 minutes to wash off any residual monomer or ungrafted polymer. The saline solution was poured off and the functionalized substrate was washed for another 30 minutes with fresh saline solution, followed by washing for 30 minutes (two times) with deionized water and then drying. As determined by mass gain, the graft density was 0.266 millimole per gram (mmol/g) of membrane.

### Preparation of Cation Exchange Nonfibrous Porous Filter Element B (FE-B)

Nylon membrane (#080ZN) was grafted with an IEM-glycine sodium salt monomer solution (0.25 M) as described in Example 30 of US Patent Application No. 2019/0194250 (Colak Atan et al.). The graft density was 0.28 mmol/g of membrane.

### Preparation of Cation Exchange Nonfibrous Porous Filter Element C (FE-C)

Nylon membrane (#080ZN) was grafted with an IEM-glycine sodium salt monomer solution as described in Example 30 of US Patent Application No. 2019/0194250 (Colak Atan et al.), but at 0.375 M concentration. The graft density was 0.38 mmol/g of membrane.

### Preparation of Cation Exchange Nonfibrous Porous Filter Element D (FE-D)

Nylon membrane (#080ZN) was grafted with a 2-[[2-methyl-2-(prop-2-enoylamino)propanoyl]amino]ethyl phosphate disodium salt (VDM-O-phosphorylethanolamine disodium salt) monomer solution using the procedure as described for FE-A, at 0.25 M concentration. The graft density was 0.12 mmol/g of membrane.

The monomer was prepared as follows: O-Phosphorylethanolamine (21.15 g) was weighed into a 500 milliliters (mL) round bottom (RB) flask, then placed in an ice-water bath with magnetic stirring. NaOH (2N (2 Normal), 150 mL) was added and the mixture was stirred until dissolved. VDM (10 mL) was added by pipette. The initially cloudy suspension was stirred for 10 minutes, by which time it had become homogeneous. A second 10 mL portion of VDM was added (total of 20 mL added). The mixture was stirred an additional 50 minutes. The reaction mixture was adjusted to pH 7 by the addition of a few drops of concentrated hydrochloric acid and filtered. % Solids=25.0%. ¹H-NMR (D₂O): δ 1.30 (s, 6H), 3.20 (t, 2H), 3.59 (q, 2H), 5.56 (dd, 1H), 5.99 (dd, 1H), 6.09 (dd, 1H).

### Preparation of Cation Exchange Nonfibrous Porous Filter Element E (FE-E)

Nylon membrane (#080ZN) was grafted with a [2-carboxy-2-[[2-methyl-2-(prop-2-enoylamino)propanoyl]amino]ethyl] phosphate disodium salt (VDM-O-phosphoserine disodium salt) monomer solution using the procedure as described for FE-A, at 0.25 M concentration. The graft density was 0.07 mmol/g of membrane.

The monomer was prepared as follows: L-O-Phosphoserine (18.5 grams) was added to a 500 mL RB flask that contained a magnetic stir bar. The flask was placed in an ice-water bath, and deionized water (40 mL) and 5N NaOH (60 mL) were added to the flask. The contents were stirred until all of the solids dissolved. After cooling in the ice bath for about 30 minutes, VDM (5.0 mL) was added by syringe. The mixture was stirred for 10 minutes and then another 8.33 mL of VDM was added (total of 13.33 mL added). Stirring was continued for 55 minutes to complete the reaction. The mixture was adjusted to pH 7 by the addition of 6 drops of concentrated hydrochloric acid. The reaction was filtered to provide the product. % Solids=28.4%. ¹H-NMR (D₂O): δ 1.34 (s, 3H), 1.38 (s, 3H), 3.81 (2m, 2H), 4.07 (m, 1H), 5.56 (dd, 1H), 5.99 (dd, 1H), 6.11 (dd, 1H).

### Preparation of Cation Exchange Nonfibrous Porous Filter Element F (FE-F)

Nylon membrane (#080ZN) was grafted with a 50:50 (mol/mol) VDM-GABA sodium salt (VDM-4-aminobutyric acid sodium salt)/VDM-phenylalanine sodium salt monomer solution as described in Example 5 of International Publication No. WO2018/048696 (Vail et al.), at 0.25 M concentration. The graft density was 0.16 mmol/g of membrane.

### Preparation of Cation Exchange Nonfibrous Porous Filter Element G (FE-G)

Nylon membrane (#080ZN) was grafted with a VDM-7-aminoheptanoic acid sodium salt monomer solution as described in Example 21 of US Patent Application No. 2019/0194250 (Colak Atan et al.), at 0.25 M concentration. The graft density was 0.18 mmol/g of membrane.

### Preparation of Cation Exchange Nonfibrous Porous Filter Element H (FE-H)

Nylon membrane (#080ZN) was grafted with a 50:50 (mol/mol) IEM-glycine sodium salt/VDM-phenylalanine sodium salt monomer solution as described in Example 11 of International Publication No. WO 2018/048696 (Vail et al.), at 0.25 M concentration. The graft density was 0.14 mmol/g of membrane.

### Preparation of Cation Exchange Nonfibrous Porous Filter Element I (FE-I)

Nylon membrane (#080ZN) was grafted with a 50:50 (mol/mol) VDM-GABA sodium salt/VDM-4-aminomethyl-cyclohexanecarboxylic acid sodium salt monomer solution as described in Example 2 of International Publication No. WO 2018/048696 (Vail et al.), at 0.25 M concentration. The graft density was 0.19 mmol/g of membrane.

### Preparation of Cation Exchange Nonfibrous Porous Filter Element J (FE-J)

Nylon membrane (#080ZN) was grafted with a 50:50 (mol/mol) IEM-glycine sodium salt/IEM-phenylalanine sodium salt monomer solution by the procedure as described for FE-H, at 0.25 M concentration. The IEM-phenylalanine sodium salt monomer was prepared as described in International Publication No. WO 2018/048696 (Vail et al.) with the exception that racemic phenylalanine was used. The graft density was 0.42 mmol/g of membrane.

### Preparation of Cation Exchange Nonfibrous Porous Filter Element K (FE-K)

Nylon membrane (#080ZN) was grafted with an IEM-glycine sodium salt monomer solution (0.25 M) as described in Example 30 of US Patent Application No. 2019/0194250 (Colak Atan et al.). The graft density was 0.26 mmol/g of membrane.

### Preparation of Salt-Tolerant Anion Exchange Nonfibrous Porous Filter Element L (FE-L)

IEM-agmatine sodium sulfate was radiation graft polymerized on nylon membrane (#080ZN) to make a guanidinyl ligand-functionalized microporous membrane similar to the procedure described in U.S. Pat. No. 10,471,398 (Bothof et al.), Example 28, and having a similar Bovine Serum Albumin (BSA) dynamic binding capacity.

### Example 1

### Flow-Through Purification Process Using Sequential Treatment of a Monoclonal Antibody Solution with an Anion Exchange Adsorptive Depth Filter, Salt-Tolerant Anion Exchange Nonfibrous Porous Filter Element, and Cation Exchange Nonfibrous Porous Filter Element.

Viral inactivated pool (VIP) of monoclonal antibody mAbA (IgG1, pI 8.0, 10.5 mg/mL, pH 6.27, 5.3 mS/cm) solution, was filtered through an anion exchange adsorptive depth filter that was prepared from the internal media components of an EMPHAZE AEX Hybrid Purifier (EMPHAZE AEX HP, obtained from the 3M Corporation) assembled in a 25 mm holder [10 bed volumes per minute (BVM)]. The challenge load was 1700 grams per liter (g/L) with a throughput of 180 liters per square meter (L/m²). Challenge load was determined as the amount of mAbA per given volume of filtration media (grams per liter, g/L). Throughput was determined as the amount of volume of liquid that passed through a given surface area of filtration media (liters per square meter, L/m²). The depth filter reduced the turbidity of the VIP solution from >60 NTU (nephelometric turbidity unit) to <5 NTU and the differential pressure did not exceed 5 psi (pounds per square inch). Table 2 shows the properties of the resulting depth filter processed solution.

An FE-L loaded challenge plate (described above) was washed with 0.9% sodium chloride (1 mL) by centrifugation at 3000 rcf (relative centrifugal force) for 5 minutes. After washing, the depth filter processed solution was filtered using the FE-L challenge plate at a 1200 g/L challenge load by centrifugation at 300 rcf for 5 minutes. The FE-L filtered solutions were pooled together. Aliquots of the pooled solution with different challenge loads [333 microliter (400 g/L challenge load) or 450 microliter (550 g/L challenge load)] were prepared for addition to the wells of a series of second challenge plates. Each second challenge plate contained two disks of a single type of cation exchange filter element selected from FE-A-FE-F (as described above). The second challenge plates were prewashed with saline solution according to the method described for FE-L challenge plates. The plates were sequentially centrifuged at 300, 600, 1200, and 3000 rcf for 5 minutes each. Filtered samples were analyzed for product and impurity analysis.

The VIP solution prior to processing had a % HMW Composition of 4.2% and an HCP concentration of 3899 nanograms per liter (ng/L).

The solution obtained after the depth filtration process step had a % HMW Composition of 4.2%, a % Monomeric mAb Yield of 98.4%, and an HCP concentration of 3366 nanograms per milliliter (ng/mL).

The pooled solution obtained after the filtration process step using FE-L had a % HMW Composition of 3.6%, a % Monomeric mAb Yield of 100.1%, and an HCP concentration of 1717 ng/mL.

The % HMW Composition, % Monomeric mAb Yield, and HCP concentration values obtained after the final process step of filtering the solution through a cation exchange filter element (selected from FE-A-FE-F) are reported in Table 3. The results are reported for experiments using either the 400 g/L challenge load or the 550 g/L challenge load.

TABLE 2
Characterization of Depth Filter processed VIP Solution

| | | Protein Concentration | |
|---|---|---|---|
| | | of mAbA | Conductivity |
| Samples | pH | (mg/mL) | (mS/cm) |
| VIP Solution before Depth | 6.27 | 10.5 | 5.3 |
| Filtration Processing | | | |
| VIP Solution after Depth | 6.22 | 10.3 | 5.7 |
| Filtration Processing | | | |

TABLE 3
% HMW Composition, % Monomeric mAb Yield, and HCP Concentration from
Purifying mAbA Solutions according to the Process of Example 1

| | Challenge Load | | | Challenge Load | | |
|---|---|---|---|---|---|---|
| | (400 g/L) | | | (550 g/L) | | |
| | | % | | | % | |
| Filter | HMW | Monomeric | HCP | HMW | Monomeric | HCP |
| Element | (%) | mAb Yield | (ng/mL) | (%) | mAb Yield | (ng/mL) |
| FE-A | 1.3 | 69.8 | 852 | 1.5 | 75.5 | 853 |
| FE-B | 2.3 | 53.9 | 728 | 2.5 | 66.0 | 774 |
| FE-C | 2.2 | 60.7 | 755 | 2.4 | 68.4 | 772 |
| FE-D | 1.1 | 56.8 | 586 | 1.4 | 67.2 | 653 |
| FE-E | 1.2 | 71.2 | 586 | 1.5 | 79.6 | 594 |
| FE-F | 2.8 | 56.6 | 791 | 3.0 | 64.1 | 826 |

### Example 2

### Flow-Through Purification Process Using Sequential Treatment of a Monoclonal Antibody Solution with an Anion Exchange Adsorptive Depth Filter, Buffer Exchange Step, Salt-Tolerant Anion Exchange Nonfibrous Porous Filter Element, and Cation Exchange Nonfibrous Porous Filter Element.

VIP of monoclonal antibody mAbA (IgG1, pI 8.0, 7.1 mg/mL, pH 6.27, 4.0 mS/cm) solution was filtered through the depth filter according to the process described in Example 1. Individual aliquots from the depth filter treated solution were adjusted to a targeted pH of either 5.5, 6.25, or 7.0 and a conductivity of either 8, 16, or 24 mS/cm. The properties of the buffer adjusted mAbA solutions are provided in Table 4. Equilibration buffers were prepared starting from 20 mM acetate buffer (pH 5.5) and adjusting with 2 M Tris. Conductivity of the buffers was adjusted using 4 M sodium chloride. Equilibration buffers were matched with the pH and conductivity properties of the buffer adjusted mAb solutions.

In the process, SARTOBIND STIC plates (primary amine functionality) were used as the anion exchange filter element challenge plates and SARTOBIND S plates (sulfonic acid functionality) were used as the cation exchange filter element challenge plates. Each plate was pre-treated by filtration with one of the equilibration buffers (selected from equilibration buffers described above). The pre-treatment filtration conditions were 500 microliters of buffer centrifuged at 1,000 rcf for 2 minutes. This process was repeated a total of 4 times. Equilibration buffer treated plates were matched to be used with mAbA solutions that were adjusted using the same buffer composition.

After equilibration, individual aliquots of buffer exchanged mAbA solution were filtered using a SARTOBIND STIC plate. The filtration conditions were 500 microliters of solution centrifuged at 1,000 rcf for 2 minutes. This process was repeated a total of 2 times for a challenge load of approximately 400 g/L (19 microliter bed volume of membrane). The resulting filtered solutions were then filtered using a SARTOBIND S plate. The filtration conditions were the same as for the SARTOBIND STIC plate. The final filtered solution was collected and analyzed. The HCP concentration, % HMW Composition, % HMW Removed, and % Monomeric mAb Yield of the final filtered solutions were determined and the results are reported in Tables 5-8.

### Comparative Example 2

During the process of Example 2, intermediate process samples of the solution were recovered after filtering through SARTOBIND STIC plates, but before filtering through the SARTOBIND S plates. The intermediate samples were analyzed for HCP concentration, % HMW Composition, % HMW Removed, and % Monomeric mAb Yield. The results are presented in Tables 5-8.

### Example 2a

The same process as described in Example 2 was followed with the exception that FE-L loaded plates were used as the anion exchange filter element challenge plates and FE-J loaded plates were used as the cation exchange filter element challenge plates. The process conditions were modified to be 1000 microliters of equilibration buffer centrifuged at 1,500 rcf for 5 minutes for each FE-L and FE-J filter plate. After equilibration, individual aliquots (500 microliters) of buffer exchanged mAbA solution were sequentially filtered with FE-L loaded plates followed by FE-J loaded plates using centrifugation at 1,500 rcf for 5 minutes. The challenge load for each well was approximately 400 g/L (9 microliter bed volume of membrane). The HCP concentration, % HMW Composition, % HMW Removed, and % Monomeric mAb Yield of the final filtered solutions were determined and the results are reported in Tables 5-8.

### Comparative Example 2a

During the process of Example 2a, intermediate process samples of the solution were recovered after filtering through FE-L loaded plates, but before filtering through the FE-J loaded plates. The intermediate samples were analyzed for HCP concentration, % HMW Composition, % HMW Removed, and % Monomeric mAb Yield. The results are presented in Tables 5-8.

### Example 2b

The same process as described in Example 2a was followed with the exception that FE-K loaded plates were used instead of FE-J plates as the cation exchange filter element challenge plates. The HCP concentration, % HMW Composition, % HMW Removed, and % Monomeric mAb Yield of the final filtered solutions were determined and the results are reported in Tables 5-8.

### Example 2c

The same process as described in Example 2 was followed with the exception that that FE-L loaded plates were used as the anion exchange filter element challenge plates and FE-F loaded plates were used as the cation exchange filter element challenge plates. The processing conditions were modified to be 1000 microliters of equilibration buffer centrifuged at 1,500 rcf for 5 minutes for each FE-L and FE-F filter plate. After equilibration, individual aliquots (500 microliters) of buffer exchanged mAbA solution were sequentially filtered with FE-L loaded plates followed by FE-F loaded plates using centrifugation at 1,500 rcf for 5 minutes. An additional centrifugation cycle (3,000 rcf for 5 minutes) was used for the FE-F loaded plates. The challenge load for each well was approximately 400 g/L (9 microliter bed volume of membrane). The HCP concentration, % HMW Composition, % HMW Removed, and % Monomeric mAb Yield of the final filtered solutions were determined and the results are reported in Tables 5-8.

TABLE 4
pH, Conductivity, mAbA Concentration, HCP Concentration,
and % HMW Composition for Buffer Adjusted Solutions
of Examples 2, 2a-c, and Comparative Examples 2 and 2a

| Buffer Adjusted | | | | | |
|---|---|---|---|---|---|
| (BA) Solution | | Measured | mAbA | | |
| (Target pH | | Conduc- | Concen- | HCP | |
| and Conduc- | Measured | tivity | tration | (ng/ | HMW |
| tivity) | pH | (mS/cm) | (mg/mL) | mL) | (%) |
| pH 5.5, 8 mS/cm | 5.5 | 7.8 | 7.1 | 930 | 5.2 |
| pH 5.5, 16 mS/cm | 5.4 | 15.7 | 7.2 | 918 | 5.2 |
| pH 5.5, 24 mS/cm | 5.4 | 24.2 | 8.6 | 915 | 5.2 |
| pH 6.25, 8 mS/cm | 6.2 | 8.0 | 7.7 | 871 | 5.1 |
| pH 6.25, 16 mS/cm | 6.2 | 16.1 | 7.4 | 873 | 5.1 |
| pH 6.25, 24 mS/cm | 6.1 | 24.7 | 8.2 | 868 | 5.1 |
| pH 7.0, 8 mS/cm | 7.0 | 8.0 | 8.6 | 839 | 4.9 |
| pH 7.0, 16 mS/cm | 7.0 | 16.2 | 8.2 | 844 | 4.9 |
| pH 7.0, 24 mS/cm | 6.9 | 24.4 | 7.8 | 826 | 4.9 |

TABLE 5
Host Cell Protein Content Following Purification by the Process
Described for Examples 2, 2a-c, and Comparative Examples 2 and 2a

| | HCP Concentration (ng/mL) | | | | | | | | |
|---|---|---|---|---|---|---|---|---|---|
| | pH 5.5 | | | pH 6.25 | | | pH 7.0 | | |
| | 8 | 16 | 24 | 8 | 16 | 24 | 8 | 16 | 24 |
| | mS/cm | mS/cm | mS/cm | mS/cm | mS/cm | mS/cm | mS/cm | mS/cm | mS/cm |
| Comparative | 794 | 828 | 801 | 794 | 756 | 775 | 750 | 797 | 787 |
| Example 2 | | | | | | | | | |
| Example 2 | 389 | 329 | 426 | 445 | 413 | 600 | 552 | 629 | 727 |
| Comparative | 242 | 209 | 232 | 205 | 172 | 187 | 188 | 211 | 208 |
| Example 2a | | | | | | | | | |
| Example 2a | 119 | 92 | 89 | 91 | 83 | 90 | 103 | 110 | 114 |
| Example 2b | 105 | 89 | 91 | 103 | 92 | 114 | 128 | 149 | 146 |
| Example 2c | 109 | 79 | 84 | 117 | 87 | 98 | 115 | 125 | 109 |

TABLE 6
HMW Composition Following Purification by the Process Described
for Examples 2, 2a-c, and Comparative Examples 2 and 2a

| | % HMW Composition | | | | | | | | |
|---|---|---|---|---|---|---|---|---|---|
| | pH 5.5 | | | pH 6.25 | | | pH 7.0 | | |
| | 8 | 16 | 24 | 8 | 16 | 24 | 8 | 16 | 24 |
| | mS/cm | mS/cm | mS/cm | mS/cm | mS/cm | mS/cm | mS/cm | mS/cm | mS/cm |
| Comparative | 4.7 | 4.7 | 4.8 | 4.8 | 4.8 | 4.9 | 5.0 | 5.0 | 5.1 |
| Example 2 | | | | | | | | | |
| Example 2 | 3.8 | 2.3 | 4.6 | 3.5 | 3.9 | 4.8 | 3.0 | 4.6 | 5.0 |
| Comparative | 1.7 | 1.0 | 1.0 | 1.8 | 1.2 | 1.0 | 2.5 | 2.2 | 1.7 |
| Example 2a | | | | | | | | | |
| Example 2a | 1.2 | 0.5 | 0.2 | 1.2 | 0.5 | 0.2 | 1.4 | 0.7 | 0.4 |
| Example 2b | 1.1 | 0.3 | 0.2 | 1.1 | 0.3 | 0.6 | 1.1 | 0.6 | 1.2 |
| Example 2c | 1.0 | 0.3 | 0.2 | 1.4 | 0.5 | 0.2 | 1.8 | 0.8 | 0.4 |

TABLE 7
HMW Removed Following Purification by the Process Described
for Examples 2, 2a-c, and Comparative Examples 2 and 2a

| | % HMW Removed | | | | | | | | |
|---|---|---|---|---|---|---|---|---|---|
| | pH 5.5 | | | pH 6.25 | | | pH 7.0 | | |
| | 8 | 16 | 24 | 8 | 16 | 24 | 8 | 16 | 24 |
| | mS/cm | mS/cm | mS/cm | mS/cm | mS/cm | mS/cm | mS/cm | mS/cm | mS/cm |
| Comparative | 12.6 | 12.1 | 10.3 | 8.9 | 7.6 | 4.7 | 3.0 | 0.5 | −3.4 |
| Example 2 | | | | | | | | | |
| Example 2 | 39.4 | 60.7 | 15.8 | 41.3 | 27.6 | 7.1 | 45.5 | 10.7 | −1.4 |
| Comparative | 69.8 | 83.1 | 83.6 | 68.6 | 80.3 | 82.3 | 58.3 | 61.5 | 70.0 |
| Example 2a | | | | | | | | | |
| Example 2a | 84.5 | 94.8 | 98.2 | 90.1 | 95.7 | 97.2 | 90.2 | 93.2 | 94.0 |
| Example 2b | 88.4 | 96.9 | 96.9 | 89.1 | 96.5 | 91.1 | 89.4 | 92.1 | 79.8 |
| Example 2c | 86.8 | 96.0 | 97.5 | 81.4 | 94.6 | 97.2 | 82.8 | 92.9 | 94.1 |

TABLE 8
Monomer Yield Following Purification by the Process Described
for Examples 2, 2a-c, and Comparative Examples 2 and 2a

| | % Monomeric mAbA Yield | | | | | | | | |
|---|---|---|---|---|---|---|---|---|---|
| | pH 5.5 | | | pH 6.25 | | | pH 7.0 | | |
| | 8 | 16 | 24 | 8 | 16 | 24 | 8 | 16 | 24 |
| | mS/cm | mS/cm | mS/cm | mS/cm | mS/cm | mS/cm | mS/cm | mS/cm | mS/cm |
| Comparative | 97.5 | 98.0 | 98.8 | 96.8 | 97.6 | 99.3 | 96.7 | 97.5 | 99.5 |
| Example 2 | | | | | | | | | |
| Example 2 | 84.1 | 93.4 | 97.5 | 86.5 | 96.0 | 98.3 | 90.3 | 96.1 | 98.4 |
| Comparative | 93.8 | 91.7 | 92.3 | 91.0 | 88.6 | 90.6 | 85.0 | 86.3 | 87.7 |
| Example 2a | | | | | | | | | |
| Example 2a | 69.2 | 52.1 | 61.8 | 44.7 | 43.4 | 70.7 | 36.3 | 48.9 | 73.5 |
| Example 2b | 56.0 | 60.6 | 82.5 | 52.5 | 65.5 | 85.4 | 48.1 | 71.3 | 84.2 |
| Example 2c | 71.7 | 65.1 | 71.3 | 70.0 | 53.2 | 70.5 | 49.1 | 45.4 | 72.5 |

### Example 3

### Flow-Through Purification Process Using Sequential Treatment of a Monoclonal Antibody Solution with an Anion Exchange Adsorptive Depth Filter, Buffer Exchange Step, Salt-Tolerant Anion Exchange Nonfibrous Porous Filter Element, and Cation Exchange Nonfibrous Porous Filter Element

VIP of monoclonal antibody mAbA (IgG1, pI 8.0, 7.1 mg/mL, pH 6.27, 4.0 mS/cm) solution was filtered through the depth filter according to the process described in Example 1. The VIP solution prior to processing had a % HMW Composition of 4.2% and an HCP concentration of 4098 ng/L. The solution obtained after the depth filtration process step had a % HMW Composition of 4.2%, a % Monomeric mAb Yield of 97.9%, and an HCP concentration of 3546 ng/mL.

Buffer adjusted mAbA solutions were prepared and the properties of the buffer adjusted mAbA solutions are provided in Table 9. Twelve different buffer adjusted mAbA solutions were prepared by adjusting the pH with 500 mM acetic acid or 2 M Tris and the conductivity using 4 M sodium chloride. The solutions had a targeted pH of either 6.0, 6.5, or 7.0 and a targeted conductivity of either 8, 14, 19, or 24 mS/cm.

In the process, FE-L loaded plates were used as the anion exchange filter element challenge plates and FE-C loaded plates were used as the cation exchange filter element challenge plates. Each plate was pre-treated by filtration with one of the equilibration buffers (selected from equilibration buffers described above in Example 2). The pre-treatment filtration conditions were 1000 microliters of buffer centrifuged at 3000 rcf for 5 minutes. Equilibration buffer treated plates were matched to be used with mAbA solutions that were adjusted using the same buffer composition. After equilibration, individual aliquots of buffer exchanged mAbA solution were filtered using an FE-L loaded plate. The filtration conditions were 1000 microliters of solution centrifuged at 300 rcf for 5 minutes. The challenge load was approximately 1200 g/L (9 microliter bed volume of membrane) and the collected filtrate was pooled for each individual condition.

The resulting filtered solutions were then filtered using an FE-C loaded plate. The filtration conditions were aliquots of the pooled solution with different challenge loads [333 microliter (400 g/L challenge load) or 450 microliters (550 g/L challenge load)] centrifuged at 100, 200, 300, 600, 1200, and 3000 rcf for 5 minutes. The final filtered solution was collected and analyzed. The HCP concentration, % HMW Composition, % Monomeric mAb Yield, % HMW Removed, and % HCP Removed of the final filtered solutions were determined and the results are reported in Tables 11-12.

### Comparative Example 3

During the process of Example 3, intermediate process samples of the solution were recovered after filtering through FE-L loaded plates, but before filtering through the FE-C loaded plates. The intermediate samples were analyzed for HCP concentration, % HMW Composition, and % Monomeric mAb Yield. The results are reported in Table 10.

### Example 3a

The same process as described in Example 3 was followed with the exception that FE-G loaded plates were used instead of FE-C plates as the cation exchange filter element challenge plates. The HCP concentration, % HMW Composition, % Monomeric mAb Yield, % HMW Removed, and % HCP Removed of the final filtered solutions were determined and the results are reported in Tables 13-14.

### Example 3b

VIP of monoclonal antibody mAbA (IgG1, pI 8.0, 7.1 mg/mL, pH 6.27, 4.0 mS/cm) solution was filtered through the depth filter according to the process described in Example 1. The VIP solution prior to processing had a % HMW Composition of 4.2% and an HCP concentration of 4098 ng/L. The solution obtained after the depth filtration process step had a % HMW Composition of 4.2%, a % Monomeric mAb Yield of 97.9%, and an HCP concentration of 3255 ng/mL. The same process as described in Example 3 was followed with the exception that FE-F loaded plates were used instead of FE-C plates as the cation exchange filter element challenge plates. The HCP concentration, % HMW Composition, % Monomeric mAb Yield, % HMW Removed, and % HCP Removed of the final filtered solutions were determined and the results are reported in Tables 15-16.

### Comparative Example 3b

During the process of Example 3b, intermediate process samples of the solution were recovered after filtering through FE-L loaded plates, but before filtering through the FE-F loaded plates. The intermediate samples were analyzed for HCP concentration, % HMW Composition, and % Monomeric mAb Yield. The results are reported in Table 10.

TABLE 9
Conductivity and pH adjustment of mAbA solutions

| | | | Measured |
|---|---|---|---|
| Buffer Adjusted | Target pH and | Measured | Conductivity |
| (BA) Solution | Conductivity | pH | (mS/cm) |
| A | pH 6.0, 8 mS/cm | 6.0 | 8.1 |
| B | pH 6.0, 14 mS/cm | 6.0 | 13.8 |
| C | pH 6.0, 19 mS/cm | 6.0 | 18.5 |
| D | pH 6.0, 24 mS/cm | 6.0 | 23.8 |
| E | pH 6.5, 8 mS/cm | 6.5 | 8.2 |
| F | pH 6.5, 14 mS/cm | 6.5 | 13.7 |
| G | pH 6.5, 19 mS/cm | 6.5 | 18.9 |
| H | pH 6.5, 24 mS/cm | 6.5 | 24.0 |
| I | pH 7.0, 8 mS/cm | 7.0 | 8.2 |
| J | pH 7.0, 14 mS/cm | 7.0 | 13.6 |
| K | pH 7.0, 19 mS/cm | 7.0 | 19.1 |
| L | pH 7.0, 24 mS/cm | 7.0 | 24.6 |

TABLE 10
% HMW Composition, HCP Concentration, and % Monomeric
mAb Yield for Comparative Examples 3 and 3b

| | BA Solution | HMW | HCP | % Monomeric |
|---|---|---|---|---|
| Example | (Table 9) | (%) | (ng/mL) | mAb Yield |
| Comparative | A | 3.5 | 1105 | 99.0 |
| Example 3 | B | 3.3 | 1127 | 99.0 |
| | C | 2.8 | 996 | 98.4 |
| | D | 2.4 | 934 | 98.2 |
| | E | 3.5 | 1062 | 99.2 |
| | F | 3.3 | 990 | 99.3 |
| | G | 2.9 | 835 | 99.1 |
| | H | 2.3 | 745 | 98.7 |
| | I | 3.4 | 980 | 98.9 |
| | J | 3.2 | 1019 | 99.4 |
| | K | 2.6 | 835 | 98.7 |
| | L | 2.3 | 698 | 97.9 |
| Comparative | A | 3.5 | 789 | 99.0 |
| Example 3b | B | 3.3 | 806 | 99.0 |
| | C | 2.8 | 730 | 98.4 |
| | D | 2.4 | 693 | 98.2 |
| | E | 3.5 | 767 | 99.2 |
| | F | 3.3 | 755 | 99.3 |
| | G | 2.9 | 714 | 99.1 |
| | H | 2.3 | 633 | 98.7 |
| | I | 3.4 | 754 | 98.9 |
| | J | 3.2 | 748 | 99.4 |
| | K | 2.6 | 688 | 98.7 |
| | L | 2.3 | 609 | 97.9 |

TABLE 11
% HMW Composition, HCP Concentration, and % Monomeric mAb Yield Following
Purification of mAbA Solutions according to the Process of Example 3

| | | Challenge Load 400 g/L | | | Challenge Load 550 g/L | | |
|---|---|---|---|---|---|---|---|
| | BA | | | % | | | % |
| | Solution | HMW | HCP | Monomeric | HMW | HCP | Monomeric |
| Example | (Table 9) | (%) | (ng/mL) | mAb Yield | (%) | (ng/mL) | mAb Yield |
| Example 3 | A | 1.3 | 630 | 58.4 | 1.7 | 682 | 72.0 |
| | B | 0.6 | 655 | 77.4 | 0.8 | 625 | 83.4 |
| | C | 0.6 | 649 | 90.2 | 0.8 | 668 | 93.2 |
| | D | 1.6 | 707 | 95.9 | 1.8 | 639 | 97.1 |
| | E | 1.1 | 828 | 70.7 | 1.4 | 803 | 77.1 |
| | F | 0.7 | 876 | 82.1 | 0.9 | 889 | 86.9 |
| | G | 0.8 | 841 | 90.4 | 1.0 | 763 | 92.7 |
| | H | 1.8 | 651 | 95.0 | 2.0 | 661 | 96.4 |
| | I | 0.9 | 995 | 74.1 | 1.1 | 1067 | 80.0 |
| | J | 0.6 | 1075 | 82.7 | 0.7 | 943 | 88.4 |
| | K | 1.0 | 827 | 91.8 | 1.2 | 775 | 93.7 |
| | L | 2.0 | 625 | 95.5 | 2.0 | 648 | 96.7 |

TABLE 12
HMW Removed and HCP Removed by the Process of Example 3

| | | Challenge load | | Challenge Load | |
|---|---|---|---|---|---|
| | BA | 400 g/L | | 550 g/L | |
| | Solution | % HMW | % HCP | % HMW | % HCP |
| Example | (Table 9) | Removed | Removed | Removed | Removed |
| Example 3 | A | 69.3 | 84.6 | 58.7 | 83.4 |
| | B | 85.0 | 84.0 | 80.9 | 84.7 |
| | C | 85.3 | 84.2 | 80.9 | 83.7 |
| | D | 60.8 | 82.7 | 57.2 | 84.4 |
| | E | 73.0 | 79.8 | 67.7 | 80.4 |
| | F | 82.5 | 78.6 | 78.1 | 78.3 |
| | G | 81.1 | 79.5 | 77.1 | 81.4 |
| | H | 56.5 | 84.1 | 53.3 | 83.9 |
| | I | 77.7 | 75.7 | 74.6 | 74.0 |
| | J | 85.3 | 73.8 | 82.3 | 77.0 |
| | K | 76.5 | 79.8 | 71.5 | 81.1 |
| | L | 53.1 | 84.7 | 51.5 | 84.2 |

TABLE 13
% HMW Composition, HCP Concentration, and % Monomeric mAb Yield Following
Purification of mAbA Solutions according to the Process of Example 3a

| | | Challenge Load 400 g/L | | | Challenge Load 550 g/L | | |
|---|---|---|---|---|---|---|---|
| | BA | | | % | | | % |
| | Solution | HMW | HCP | Monomeric | HMW | HCP | Monomeric |
| Example | (Table 9) | (%) | (ng/mL) | mAb Yield | (%) | (ng/mL) | mAb Yield |
| Example 3a | A | 1.9 | 762 | 53.9 | 2.3 | 782 | 66.7 |
| | B | 1.7 | 733 | 58.3 | 2.2 | 839 | 69.8 |
| | C | 1.7 | 661 | 63.3 | 2.0 | 720 | 76.9 |
| | D | 1.2 | 584 | 66.6 | 1.3 | 654 | 74.0 |
| | E | 1.9 | 781 | 53.5 | 1.9 | 684 | 47.7 |
| | F | 1.7 | 787 | 56.5 | 1.9 | 667 | 66.6 |
| | G | 1.6 | 646 | 65.0 | 1.7 | 605 | 82.8 |
| | H | 1.2 | 510 | 68.4 | 1.4 | 524 | 74.6 |
| | I | 1.1 | 618 | 30.4 | 1.9 | 784 | 61.7 |
| | J | 1.9 | 699 | 63.8 | 2.1 | 793 | 80.0 |
| | K | 1.5 | 627 | 72.1 | 1.7 | 694 | 79.3 |
| | L | 1.1 | 477 | 72.1 | 1.2 | 500 | 77.2 |

TABLE 14
HMW Removed and HCP Removed by the Process of Example 3a

| | | Challenge Load | | Challenge Load | |
|---|---|---|---|---|---|
| | BA | 400 g/L | | 550 g/L | |
| | Solution | % HMW | % HCP | % HMW | % HCP |
| Example | (Table 9) | Removed | Removed | Removed | Removed |
| Example 3a | A | 53.5 | 81.4 | 46.2 | 80.9 |
| | B | 58.8 | 82.1 | 46.8 | 79.5 |
| | C | 60.2 | 83.9 | 51.6 | 82.4 |
| | D | 71.7 | 85.7 | 69.5 | 84.0 |
| | E | 55.1 | 80.9 | 55.3 | 83.3 |
| | F | 60.0 | 80.8 | 54.0 | 83.7 |
| | G | 62.6 | 84.2 | 60.5 | 85.2 |
| | H | 71.5 | 87.5 | 65.6 | 87.2 |
| | I | 73.6 | 84.9 | 53.7 | 80.9 |
| | J | 54.3 | 83.0 | 50.7 | 80.6 |
| | K | 65.1 | 84.7 | 58.8 | 83.1 |
| | L | 73.3 | 88.4 | 70.4 | 87.8 |

TABLE 15
% HMW Composition, HCP Concentration, and % Monomeric mAb Yield Following
Purification of mAbA Solutions according to the Process of Example 3b

| | | Challenge Load 400 g/L | | | Challenge Load 550 g/L | | |
|---|---|---|---|---|---|---|---|
| | BA | | | % | | | % |
| | Solution | HMW | HCP | Monomeric | HMW | HCP | Monomeric |
| Example | (Table 9) | (%) | (ng/mL) | mAb Yield | (%) | (ng/mL) | mAb Yield |
| Example 3b | A | 2.2 | 531 | 53.1 | 2.5 | 643 | 66.3 |
| | B | 1.5 | 390 | 52.9 | 1.6 | 536 | 66.5 |
| | C | 0.7 | 454 | 62.9 | 1.0 | 485 | 72.2 |
| | D | 0.3 | 475 | 71.6 | 0.5 | 510 | 80.6 |
| | E | 2.3 | 462 | 62.6 | 2.3 | 587 | 68.4 |
| | F | 1.3 | 545 | 71.0 | 2.2 | 468 | 73.0 |
| | G | 0.8 | 513 | 73.8 | 1.1 | 467 | 74.8 |
| | H | 0.3 | 416 | 74.8 | 0.4 | 358 | 80.9 |
| | I | 2.1 | 534 | 50.0 | 1.8 | 507 | 49.4 |
| | J | 1.3 | 481 | 60.0 | 1.9 | 529 | 73.8 |
| | K | 0.6 | 420 | 66.6 | 1.1 | 476 | 76.3 |
| | L | 0.4 | 425 | 76.8 | 0.4 | 414 | 82.3 |

TABLE 16
HMW Removed and HCP Removed by the Process of Example 3b

| | | Challenge Load | | Challenge Load | |
|---|---|---|---|---|---|
| | BA | 400 g/L | | 550 g/L | |
| | Solution | % HMW | % HCP | % HMW | % HCP |
| Example | (Table 9) | Removed | Removed | Removed | Removed |
| Example 3b | A | 48.4 | 85.3 | 40.4 | 82.3 |
| | B | 63.0 | 89.2 | 61.7 | 85.2 |
| | C | 83.0 | 87.5 | 75.2 | 86.6 |
| | D | 92.3 | 86.9 | 88.3 | 85.9 |
| | E | 46.2 | 87.3 | 44.7 | 83.8 |
| | F | 68.6 | 85.0 | 46.4 | 87.1 |
| | G | 80.7 | 85.8 | 74.8 | 87.1 |
| | H | 91.8 | 88.5 | 89.9 | 90.1 |
| | I | 48.9 | 85.3 | 55.8 | 86.0 |
| | J | 69.2 | 86.7 | 54.3 | 85.4 |
| | K | 86.6 | 88.4 | 74.7 | 86.9 |
| | L | 91.2 | 88.3 | 90.3 | 88.6 |

### Example 4

### Flow-Through Purification Process Using Sequential Treatment of a Monoclonal Antibody Solution with a Buffer Exchange Step, Salt-Tolerant Anion Exchange Nonfibrous Porous Filter Element, and Cation Exchange Nonfibrous Porous Filter Element

Pooled monoclonal antibody mAb2 (IgG1, pI about 8), was purified using a ZetaPlus ZP90 depth filter (3M Corporation) followed by a protein A chromatography step (mAbSelect ProA resin, GE Healthcare Life Sciences, Pittsburgh, Pa.) with an acetic acid elution and a 30 minute hold at pH 3.5 for viral inactivation. Following viral inactivation, the resulting low pH solution was neutralized with 2 M Tris. The neutralized solution had an initial pH of 7.4 and a conductivity (mS/cm) of 10.2.

Buffer adjusted mAb2 solutions were prepared and the properties of the buffer adjusted mAb2 solutions are provided in Table 17. Nine different buffer adjusted mAb2 solutions were prepared by adjusting the pH with 500 mM acetic acid and the conductivity using 4 M sodium chloride. The solutions had a targeted pH of either 6.0, 6.5, or 7.0 and a targeted conductivity of either 12, 17, or 22 mS/cm.

In the process, FE-L loaded plates were used as the anion exchange filter element challenge plates and FE-C loaded plates were used as the cation exchange filter element challenge plates. Each plate was pre-treated by filtration with one of the equilibration buffers (selected from equilibration buffers described above in Example 2). The pre-treatment filtration conditions were 1000 microliters of buffer centrifuged at 3000 rcf for 5 minutes. Equilibration buffer treated plates were matched to be used with mAb2 solutions that were adjusted using the same buffer composition. After equilibration, individual aliquots of buffer exchanged mAb2 solution were filtered using an FE-L loaded plate. The filtration conditions were 1000 microliters of solution centrifuged at 300 rcf for 5 minutes. The challenge load was approximately 350 g/L (9 microliter bed volume of membrane) and the collected filtrate was pooled for each individual condition.

The resulting filtered solutions were then filtered using an FE-C loaded plate. The filtration conditions were 1000 microliters of solution centrifuged at 300, 600, 1200, 3000 rcf for 5 minutes. The final filtered solution was collected and analyzed. The HCP concentration, % HMW Composition, % HMW Removed, and % Monomeric mAb Yield of the final filtered solutions were determined and the results are reported in Tables 18-21.

### Comparative Example 4

During the process of Example 4, intermediate process samples of the solution were recovered after filtering through FE-L loaded plates, but before filtering through the FE-C loaded plates. The intermediate samples were analyzed for HCP concentration, % HMW Composition, % HMW Removed, and % Monomeric mAb Yield. The results are presented in Tables 18-21.

### Example 4a

The same process as described in Example 4 was followed with the exception that FE-F loaded plates were used instead of FE-C plates as the cation exchange filter element challenge plates. The HCP concentration, % HMW Composition, % HMW Removed, and % Monomeric mAb Yield of the final filtered solutions were determined and the results are reported in Tables 18-21.

### Example 4b

The same process as described in Example 4 was followed with the exception that FE-E loaded plates were used instead of FE-C plates as the cation exchange filter element challenge plates. The HCP concentration, % HMW Composition, % HMW Removed, and % Monomeric mAb Yield of the final filtered solutions were determined and the results are reported in Tables 18-21.

### Example 4c

The same process as described in Example 4 was followed with the exception that FE-H loaded plates were used instead of FE-C plates as the cation exchange filter element challenge plates. The HCP concentration, % HMW Composition, % HMW Removed, and % Monomeric mAb Yield of the final filtered solutions were determined and the results are reported in Tables 18-21.

### Example 4d

The same process as described in Example 4 was followed with the exception that FE-I loaded plates were used instead of FE-C plates as the cation exchange filter element challenge plates. The HCP concentration, % HMW Composition, % HMW Removed, and % Monomeric mAb Yield of the final filtered solutions were determined and the results are reported in Tables 18-21.

TABLE 17
Characterization of mAb2 Solutions after Depth Filtration
and Protein A Purification (Example 4)

| | | Measured | | |
|---|---|---|---|---|
| mAb2 Solution | Measured | Conductivity | HCP | HMW |
| Conditions | pH | (mS/cm) | (ng/mL) | (%) |
| pH 6.0, 12 mS/cm | 6.0 | 12.1 | 332 | 4.1 |
| pH 6.0, 17 mS/cm | 6.0 | 17.6 | 387 | 4.1 |
| pH 6.0, 22 mS/cm | 6.0 | 22.5 | 388 | 4.0 |
| pH 6.5, 12 mS/cm | 6.5 | 12.1 | 407 | 4.5 |
| pH 6.5, 17 mS/cm | 6.5 | 17.4 | 389 | 4.4 |
| pH 6.5, 22 mS/cm | 6.5 | 22.6 | 460 | 4.4 |
| pH 7.0, 12 mS/cm | 7.0 | 12.1 | 440 | 4.9 |
| pH 7.0, 17 mS/cm | 7.0 | 17.3 | 468 | 4.8 |
| pH 7.0, 22 mS/cm | 7.0 | 22.7 | 411 | 4.7 |

TABLE 18
Host Cell Protein Content Following Purification by the Process
Described for Examples 4, 4a-4d, and Comparative Example 4

| | HCP Concentration (ng/mL) | | | | | | | | |
|---|---|---|---|---|---|---|---|---|---|
| | pH 6.0 | | | pH 6.5 | | | pH 7.0 | | |
| | 12 | 17 | 22 | 12 | 17 | 22 | 12 | 17 | 22 |
| | mS/cm | mS/cm | mS/cm | mS/cm | mS/cm | mS/cm | mS/cm | mS/cm | mS/cm |
| Comparative | 48 | 82 | 106 | 29 | 57 | 86 | 34 | 40 | 63 |
| Example 4 | | | | | | | | | |
| Example 4 | 16 | 50 | 66 | 13 | 31 | 58 | 14 | 18 | 43 |
| Example 4a | 22 | 50 | 65 | 14 | 27 | 60 | 15 | 22 | 49 |
| Example 4b | 25 | 61 | 75 | 16 | 36 | 63 | 15 | 22 | 41 |
| Example 4c | 22 | 64 | 70 | 16 | 38 | 68 | 15 | 22 | 49 |
| Example 4d | 25 | 67 | 78 | 14 | 32 | 67 | 16 | 18 | 41 |

TABLE 19
HMW Composition Following Purification by the Process Described
for Examples 4, 4a-4d, and Comparative Example 4

| | % HMW Composition | | | | | | | | |
|---|---|---|---|---|---|---|---|---|---|
| | pH 6.0 | | | pH 6.5 | | | pH 7.0 | | |
| | 12 | 17 | 22 | 12 | 17 | 22 | 12 | 17 | 24 |
| | mS/cm | mS/cm | mS/cm | mS/cm | mS/cm | mS/cm | mS/cm | mS/cm | mS/cm |
| Comparative | 2.3 | 2.1 | 2.1 | 1.8 | 1.9 | 1.8 | 1.7 | 1.8 | 1.6 |
| Example 4 | | | | | | | | | |
| Example 4 | 1.4 | 2.1 | 1.9 | 1.9 | 1.7 | 1.7 | 1.7 | 1.5 | 1.8 |
| Example 4a | 1.0 | 1.5 | 1.8 | 1.0 | 1.5 | 1.9 | 1.4 | 1.5 | 1.8 |
| Example 4b | 2.0 | 2.0 | 2.0 | 1.8 | 1.9 | 1.7 | 1.8 | 1.8 | 1.5 |
| Example 4c | 1.2 | 1.8 | 1.9 | 1.3 | 1.7 | 1.7 | 1.2 | 1.5 | 1.8 |
| Example 4d | 1.4 | 2.1 | 2.0 | 1.7 | 1.7 | 1.8 | 1.6 | 1.6 | 1.5 |

TABLE 20
HMW Removed by the Process Described for Examples
4, 4a-4d, and Comparative Example 4

| | % HMW Removed | | | | | | | | |
|---|---|---|---|---|---|---|---|---|---|
| | pH 6.0 | | | pH 6.5 | | | pH 7.0 | | |
| | 12 | 17 | 22 | 12 | 17 | 22 | 12 | 17 | 22 |
| | mS/cm | mS/cm | mS/cm | mS/cm | mS/cm | mS/cm | mS/cm | mS/cm | mS/cm |
| Comparative | 48.0 | 53.7 | 54.2 | 61.7 | 59.2 | 59.9 | 65.9 | 63.0 | 66.5 |
| Example 4 | | | | | | | | | |
| Example 4 | 69.8 | 54.3 | 58.3 | 59.9 | 62.6 | 63.1 | 65.4 | 68.6 | 62.7 |
| Example 4a | 80.9 | 69.6 | 60.6 | 80.5 | 69.2 | 59.7 | 73.2 | 68.8 | 61.7 |
| Example 4b | 57.6 | 55.7 | 56.0 | 62.7 | 59.5 | 64.5 | 63.9 | 61.7 | 68.0 |
| Example 4c | 74.3 | 60.2 | 58.5 | 74.4 | 62.7 | 62.9 | 76.0 | 69.0 | 60.8 |
| Example 4d | 70.2 | 53.8 | 55.9 | 63.7 | 64.3 | 61.3 | 66.6 | 66.4 | 67.5 |

TABLE 21
Monomer Yield Following the Process Described for
Examples 4, 4a-4d, and Comparative Example 4

| | % Monomeric mAb2 Yield | | | | | | | | |
|---|---|---|---|---|---|---|---|---|---|
| | pH 6.0 | | | pH 6.5 | | | pH 7.0 | | |
| | 12 | 17 | 22 | 12 | 17 | 22 | 12 | 17 | 22 |
| | mS/cm | mS/cm | mS/cm | mS/cm | mS/cm | mS/cm | mS/cm | mS/cm | mS/cm |
| Comparative | 93.5 | 92.7 | 93.0 | 89.0 | 90.6 | 91.1 | 86.7 | 88.7 | 89.0 |
| Example 4 | | | | | | | | | |
| Example 4 | 88.5 | 92.1 | 91.9 | 89.0 | 90.2 | 90.6 | 87.1 | 87.8 | 89.4 |
| Example 4a | 82.0 | 87.9 | 90.1 | 81.9 | 87.6 | 90.1 | 82.7 | 86.9 | 89.4 |
| Example 4b | 90.6 | 92.0 | 91.7 | 88.2 | 90.5 | 88.5 | 86.4 | 88.6 | 88.4 |
| Example 4c | 87.7 | 91.0 | 91.5 | 85.8 | 89.6 | 89.6 | 85.3 | 86.6 | 89.7 |
| Example 4d | 88.7 | 92.5 | 92.5 | 87.6 | 89.2 | 90.6 | 86.4 | 87.7 | 88.3 |

The complete disclosures of the patents, patent documents, and publications cited herein are incorporated by reference in their entirety as if each were individually incorporated. To the extent that there is any conflict or discrepancy between this specification as written and the disclosure in any document that is incorporated by reference herein, this specification as written will control. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A flow-through process for purifying a target molecule from a biological solution in a sample that includes the target molecule, the process comprising:
optionally, contacting the sample with an anion exchange adsorptive depth filter;
optionally, conducting a buffer exchange with the sample before and/or after contacting the sample with an anion exchange adsorptive depth filter;
contacting the sample with a salt-tolerant anion exchange nonfibrous porous filter element; and
immediately thereafter, contacting the sample with a cation exchange nonfibrous porous filter element;
wherein the flow-through process comprises one or two buffer exchanges, with no buffer exchange between the sample contacting the salt-tolerant anion exchange nonfibrous porous filter element and the cation exchange nonfibrous porous filter element.

2. A flow-through process for purifying a target molecule from a biological solution in a sample that includes the target molecule, the process comprising:
contacting the sample with an anion exchange adsorptive depth filter;
immediately thereafter, contacting the sample with a salt-tolerant anion exchange nonfibrous porous filter element; and
immediately thereafter, contacting the sample with a cation exchange nonfibrous porous filter element;
wherein the flow-through process comprises no buffer exchanges.

3. A flow-through process for purifying a target molecule from a biological solution in a sample that includes the target molecule, the process comprising:
contacting the sample with an anion exchange adsorptive depth filter;
conducting a buffer exchange with the sample after contacting the sample with an anion exchange adsorptive depth filter;
contacting the sample with a salt-tolerant anion exchange nonfibrous porous filter element; and
immediately thereafter, contacting the sample with a cation exchange nonfibrous porous filter element.

4. A flow-through process for purifying a target molecule from a biological solution in a sample that includes the target molecule, the process comprising:
conducting a buffer exchange with the sample;
contacting the sample with a salt-tolerant anion exchange nonfibrous porous filter element; and
immediately thereafter, contacting the sample with a cation exchange nonfibrous porous filter element.

5. The flow-through process of claim 1 wherein the target molecule comprises a monoclonal antibody.

6. The flow-through process of claim 1 wherein the biological solution comprises a neutralized viral inactivation pool.

7. The flow-through process of claim 1 wherein the anion exchange adsorptive depth filter comprises a porous substrate comprising immobilized anion exchange ligands.

8. The flow-through process of claim 7 wherein the anion exchange ligands of the anion exchange adsorptive depth filter comprise cationic nitrogen-containing ligands.

9. The flow-through process of claim 8 wherein the cationic nitrogen-containing ligands of the anion exchange adsorptive depth filter comprise a primary amine, a secondary amine, a tertiary amine, or combinations thereof.

10. The flow-through process of claim 9 wherein the cationic nitrogen-containing ligands of the anion exchange adsorptive depth filter comprise a quaternary ammonium-containing ligand, a guanidinyl-containing ligand, or a combination thereof.

11. The flow-through process of claim 7 wherein the anion exchange adsorptive depth filter comprises a porous substrate and a ligand-functional polymer grafted thereto, wherein the grafted ligand-functional polymer is of the formula:
-(M^{PI})_{w}-(M^{b})ₓ-(M_{c})_{y}-(M^{d})_{z},
wherein:
-(M^{PI})_{w} represent the residue of grafted photoinitiator monomers, where w is zero or at least one;
-(M^{b})ₓ represents polymerized ligand monomers, having “x” polymerized monomer units, where x is at least one, and the ligand monomer is of the formula (X): 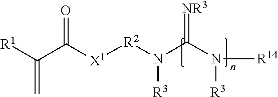
wherein:
R¹ is H or CH₃;
R² is a (hetero)hydrocarbylene;
each R³ is independently H or (hetero)hydrocarbyl;
R¹⁴ is H, (hetero)hydrocarbyl, or —N(R³)₂, where each R³ is independently H or (hetero)hydrocarbyl;
X¹ is —O— or —NR³—, where R³ is H or (hetero)hydrocarbyl, and
n is 1 or 2;
-(M^{c})_{y} represents polymerized crosslinking monomers, having y polymerized monomer units, where y may be zero or at least one; and
-(M^{d})_{z} represents polymerized hydrophilic monomers, having z polymerized monomer units, where z may be zero or at least one.

12. The flow-through process of claim 1 wherein the salt-tolerant anion exchange nonfibrous porous filter element comprises a porous membrane comprising immobilized anion exchange ligands.

13. The flow-through process of claim 12 wherein the anion exchange ligands of the salt-tolerant anion exchange nonfibrous porous filter element comprise cationic nitrogen-containing ligands.

14. The flow-through process of claim 13 wherein the cationic nitrogen-containing ligands of the salt-tolerant anion exchange nonfibrous porous filter element comprise guanidinyl-containing ligands.

15. The flow-through process of claim 14 wherein the grafted copolymer of the salt-tolerant anion exchange nonfibrous porous filter element comprises interpolymerized monomer units comprising:
a guanidinyl-containing ligand monomer;
an amide monomer;
an oxy monomer selected from the group of epoxy functional monomer units, alkyl ether functional monomer units, and combinations thereof; and
a poly(alkylene oxide) monomer.

16. The flow-through process of claim 12 wherein the salt-tolerant anion exchange nonfibrous porous filter element comprises a nonfibrous porous filter element and a ligand-functional polymer grafted thereto, wherein the grafted ligand-functional polymer is of the formula:
-(M^{PI})_{w}-(M^{b})ₓ-(M^{c})_{y}-(M_{d})_{z},
wherein:
-(M^{PI})_{w}- represent the residue of grafted photoinitiator monomers, where w is zero or at least one;
-(M^{b})ₓ represents polymerized ligand monomers, having “x” polymerized monomer units, where x is at least one, and the ligand monomer is of the formula (X): 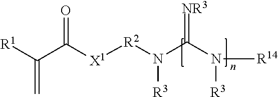
wherein:
R¹ is H or CH₃;
R² is a (hetero)hydrocarbylene;
each R³ is independently H or (hetero)hydrocarbyl;
R¹⁴ is H, (hetero)hydrocarbyl, or —N(R³)₂, where each R³ is independently H or (hetero)hydrocarbyl;
X¹ is —O— or —NR³—, where R³ is H or (hetero)hydrocarbyl, and
n is 1 or 2;
-(M^{c})_{y} represents polymerized crosslinking monomers, having y polymerized monomer units, where y may be zero or at least one; and
-(M^{d})_{z} represents polymerized hydrophilic monomers, having z polymerized monomer units, where z may be zero or at least one.

17. The flow-through process of claim 1 wherein the cation exchange nonfibrous porous filter element comprises:
a nonfibrous porous substrate; and
disposed on the porous nonfibrous membrane, a polymer comprising:
a hydrocarbon backbone and a plurality of pendant groups attached to the hydrocarbon backbone, wherein each of a first plurality of pendant groups comprises:
at least one acidic group or salt thereof; and
a spacer group that directly links the at least one acidic group or salt thereof to the hydrocarbon backbone by a chain of at least 6 catenated atoms.

18. The flow-through process of claim 17 wherein the polymer that is covalently attached to the nonfibrous porous filter element is a copolymer covalently attached to the nonfibrous porous filter element comprises a reaction product of a monomer composition comprising:
a first monomer comprising:
at least one ethylenically unsaturated group;
at least one acidic group or salt thereof; and
a spacer group that directly links the at least one ethylenically unsaturated group and the at least one acidic group or salt thereof by a chain of at least 6 catenated atoms; and
a second monomer comprising:
at least one ethylenically unsaturated group;
at least one acidic group or salt thereof; and
a spacer group that directly links the at least one ethylenically unsaturated group and the at least one acidic group by a chain of at least 6 catenated atoms;
wherein the second monomer is different than the first monomer; and
wherein a mole ratio of the first monomer to the second monomer is in a range of 95:5 to 5:95.

19. A filter cartridge comprising salt-tolerant anion exchange nonfibrous porous filter element and a cation exchange nonfibrous porous filter element;
wherein the salt-tolerant anion exchange nonfibrous porous filter element comprises:
a nonfibrous porous filter element comprising immobilized cationic nitrogen-containing ligands, or
a nonfibrous porous filter element and a ligand-functional polymer grafted thereto, wherein the grafted ligand-functional polymer is of the formula:
-(M^{PI})_{w}-(M^{b})ₓ-(M^{c})_{y}-(M^{d})_{z},
wherein:
-(M^{PI})_{w}- represent the residue of grafted photoinitiator monomers, where w is zero or at least one;
-(M^{b})ₓ represents polymerized ligand monomers, having “x” polymerized monomer units, where x is at least one, and the ligand monomer is of the formula (X): 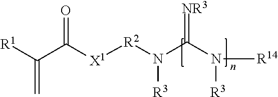
wherein:
R¹ is H or CH₃;
R² is a (hetero)hydrocarbylene;
each R³ is independently H or (hetero)hydrocarbyl;
R¹⁴ is H, (hetero)hydrocarbyl, or —N(R³)₂, where each R³ is independently H or (hetero)hydrocarbyl;
X¹ is —O— or —NR³—, where R³ is H or (hetero)hydrocarbyl, and
n is 1 or 2;
-(M^{c})_{y} represents polymerized crosslinking monomers, having y polymerized monomer units, where y may be zero or at least one; and
-(M^{d})_{z} represents polymerized hydrophilic monomers, having z polymerized monomer units, where z may be zero or at least one; and
wherein the cation exchange nonfibrous porous filter element comprises:
a nonfibrous porous filter element; and
disposed on the nonfibrous porous filter element, a polymer comprising:
a hydrocarbon backbone and a plurality of pendant groups attached to the hydrocarbon backbone, wherein each of a first plurality of pendant groups comprises:
at least one acidic group or salt thereof; and
a spacer group that directly links the at least one acidic group or salt thereof to the hydrocarbon backbone by a chain of at least 6 catenated atoms.

20. The filter cartridge of claim 19 wherein the cation exchange nonfibrous porous filter element is positioned downstream from the salt-tolerant anion exchange nonfibrous porous filter element.
